(19) United States
(12) Patent Application Publication
(10) Pub. No.: US 2021/0030798 A1
(43) Pub. Date: Feb. 4, 2021

(54) CHIMERIC RECEPTOR

(71) Applicant: AUTOLUS LIMITED, London (GB)

(72) Inventors: Martin Pulé, London (GB); Shaun Cordoba, London (GB); Shimobi Onuoha, London (GB); Simon Thomas, London (GB); Evangelia Kokalaki, London (GB); Ram Jha, London (GB)

(21) Appl. No.: 16/967,377

(22) PCT Filed: Feb. 4, 2019
(86) PCT No.: PCT/GB2019/050294

§ 371 Date: Aug. 4, 2020

(30) Foreign Application Priority Data
Feb. 5, 2018 (GB) 1801831.7

(51) Int. Cl. A61K 35/17; C07K 14/725; C07K 16/28; C07K 14/705

(52) U.S. Cl. CPC A61K 35/17; C07K 14/7051; C07K 16/2827; A61K 2039/5156; C07K 16/289; C07K 14/70578; C07K 16/2863

(57) ABSTRACT

The present invention provides a chimeric receptor which binds a target antigen on a target cell, which comprises: a first antigen binding domain which binds a first epitope of the target antigen, a second antigen binding domain which binds a second epitope of the target antigen; a transmembrane domain; and an intracellular signalling domain.

### FIELD OF THE INVENTION

The present invention relates to a chimeric receptor comprising two or more antigen binding domains. In particular, it relates to a chimeric receptor having binding domains which can concatenate target antigen at a T-cell:target cell synapse.

### BACKGROUND TO THE INVENTION

Chimeric Antigen Receptors (CARs)

A number of immunotherapeutic agents have been described for use in cancer treatment, including therapeutic monoclonal antibodies (mAbs), bi-specific T-cell engagers and chimeric antigen receptors (CARs).

Chimeric antigen receptors are proteins which graft the specificity of a monoclonal antibody (mAb) to the effector function of a T-cell. Their usual form is that of a type I transmembrane domain protein with an antigen recognizing amino terminus (binder), and a transmembrane domain connected to an endodomain which transmits T-cell activation signals.

The most common form of these molecules are fusions of single-chain variable fragments (scFv) derived from monoclonal antibodies, which recognize a target antigen, fused via a trans-membrane domain to a signalling endodomain. Such molecules result in activation of the T-cell in response to recognition by the scFv of its target. When T cells express such a CAR, they recognize and kill target cells that express the target antigen. CARs have been developed against various tumour-associated antigens and many are currently undergoing clinical trials.

Although CAR-T cell-mediated treatment have shown success towards abundant target antigens such as CD19 or GD2, chimeric antigen receptors have been reported to fail to signal in response to very low-density antigens.

For example, a CAR-T study targeting anaplastic lymphoma kinase (ALK), showed that the CAR-T cells had limited anti-tumor efficacy in two xenograft models of human neuroblastoma. It was shown that cytokine production was highly dependent upon ALK target density and that target density of ALK on neuroblastoma cell lines was insufficient for maximal activation of CAR T cells (Walker et al. (2017) Mol. Ther. 25, 2189-2201).

Another study involved the use of anti-CD22 CAR-T cell in the treatment of relapsed and/or refractory pre-B cell acute lymphoblastic leukemia (B-ALL), although dose-dependent antileukemic activity was observed, some relapses were observed. Relapses were associated with diminished CD22 site density that were thought to permitted CD22+ cell escape from killing by CD22-CAR T cells (Fry et al. (2017) Nat. Med. doi:10.1038/nm.4441).

There is a hierarchy of CAR T-cell activation from killing, to cytokine release to proliferation. A CAR T-cell may kill a target cell with low density antigen but fail to fully activate.

Another issue with CAR-T cell therapies is that CAR-T cells often fail to signal in response to cells that express long or bulky surface antigens. An optimum synaptic distance is required for efficient triggering of downstream signalling after antigen encounter. When the synapse length is short phosphatases such as CD45 and CD148, which have large ectodomains, are excluded and allow tyrosine phosphorylation to be initiated in the absence of these negative regulators. Smaller antigens such as CD19 do not provide a barrier to optimum synapse formation and can be targeted efficiently at multiple epitopes. Large proteins such as CD22 and CD21, pose a unique problem. Targeting a membrane distal epitope on such proteins may provide a suboptimal synapse length allowing phosphatases to enter the synapse and inhibit tyrosine phosphorylation (see FIG. 1). Targeting membrane proximal regions may be hindered by steric occlusion of the epitope.

As mentioned above, ligation of low density antigens also results in poor synapse formation and thus may permit the presence of phosphatases within the synapse dampening tyrosine phosphorylation, kinase activity and thus CAR signalling. Instances in which both the antigen density is low and the target antigen is large, such as CD22 on the surface of B cells, are particularly challenging for CAR T cell therapy.

There is therefore a need for alternative CAR T-cell approaches, capable of killing target cells expressing a low density of target antigen and/or expressing a large or bulky target antigen.

### DESCRIPTION OF THE FIGURES

FIG. 1—Schematic diagram illustrating a tumour cell:CAR-T cell synapse where the chimeric antigen receptor binds to a large target antigen (in this case CD22). In a successful T-cell:target cell synapse, phosphatases such as CD45 and CD148, which have large ectodomains, are excluded and allow tyrosine phosphorylation to occur and a signal to be propagated. Targeting a long antigen, such as CD22 may give rise to an excessive synapse length allowing phosphatases to enter the synapse and inhibit tyrosine phosphorylation.

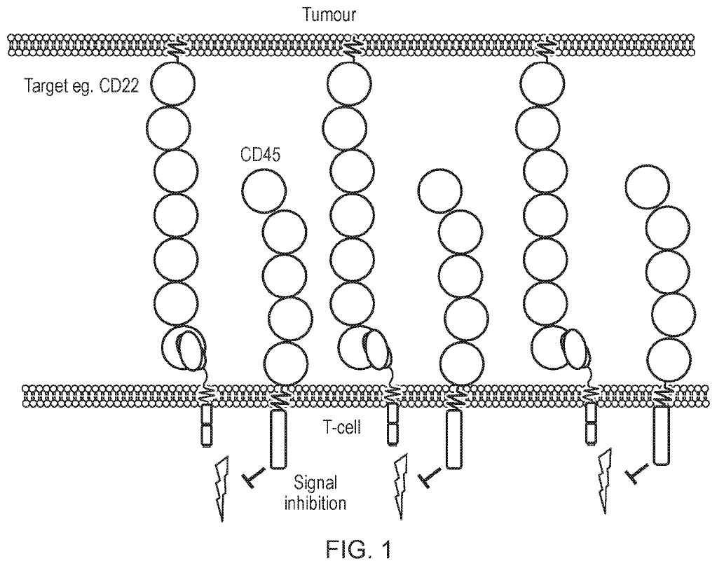

FIG. 2—Schematic diagram illustrating a tumour cell:CAR-T cell synapse where the chimeric antigen receptor binds to a large target antigen (in this case CD22) but has two antigen binding domains targeting different epitopes of the target antigen. One antigen-binding domain targeting domain targets a membrane proximal epitope and the other antigen binding domain targets a membrane distal epitope. Targeting two distinct epitopes has the effect of “levering down” and flattening the CD22 target antigen which reduces the overall synapse distance, thereby more effectively excluding phosphatases such as CD45 and CD148 from the synapse.

FIG. 3—Schematic diagram illustrating how linker length and properties can be selected to avoid intramolecular binding

FIG. 4—Schematic diagram illustrating three Fab-based bivalent chimeric receptor designs according to the present invention. A: Fab scFv; B: Dual Fab; C: Dual Fab scFv; and D: Coiled-coil spacer CAR

FIG. 5—Schematic diagram illustrating three further Fab-based bivalent chimeric receptors. A: an alternative dual Fab chimeric receptor arrangement in which the two antigen-binding domains have the same light chain variable domain, but different heavy chain variable domains; B: Fab dAb; C: Dual Fab dAb.

FIG. 6—Schematic diagram illustrating hybrid chimeric receptors which have both tanCAR and Fab-based elements. A: scFv tanFab; B: dAb tanFab C: dual variable tanFab

FIG. 7—Schematic diagram illustrating three Fc-based bivalent chimeric receptors, based on non-conventional constant domain association. A: Knob in holes Fc Dual-scFv; B: Strand exchange Fc Dual-scFv; C: Charge pair Fc Dual-scFv

FIG. 8—Schematic diagram illustrating three CH3-based bivalent chimeric receptors, based on non-conventional constant domain association. A: Knob in holes Fc Dual-scFv; B: Strand exchange Fc Dual-scFv; C: Charge pair Fc Dual-scFv

FIG. 9—Schematic diagram illustrating two alternative formats for bivalent chimeric receptors; A: Leucine zipper Dual-scFv; B: CD79a/b Dual-scFv.

FIG. 10—Schematic diagram illustrating two bivalent chimeric receptor designs according to the present invention. A: A tanCAR having scFv antigen-binding domains; B: a tanCAR having domain antibody (dAb) antigen-binding domains.

FIG. 11—CD22 specific cytotoxicity of 1D9/10C1 ConCAT CAR T cells compared to 1D9 CAR and 10C1 CAR. A) construct diagram of 1D9 CAR, 10C1 CAR and 1D9/10C1 ConCAT CAR; B) Cytotoxicity assay of CAR-T cells with control (SupT1-NT) or CD22-expressing (SupT1-CD22+) target cells.

FIG. 12—CD22 specific cytotoxicity of g5_44/10C1 ConCAT CAR T cells compared to g5_44 CAR and 10C1 CAR. A) construct diagram of g5_44 CAR, 10C1 CAR and g5_44/10C1 ConCAT CAR; B) Cytotoxicity assay of CAR-T cells with control (SupT1-NT) or CD22-expressing (SupT1-CD22+) target cells.

### SUMMARY OF ASPECTS OF THE INVENTION

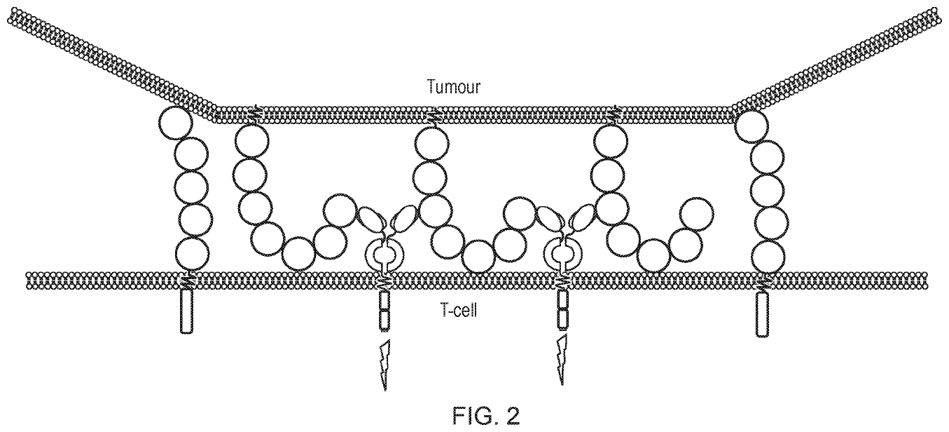

The invention relates to chimeric receptors which can concatenate target antigen on the cell surface (FIG. 2). This approach can be used to increase the sensitivity of CAR T cells when targeting difficult antigens and/or in situations where antigen density is low. The chimeric receptor comprises two antigen binding domains which target different epitopes of the same antigen.

Thus in a first aspect the present invention provides a chimeric receptor which binds a target antigen on a target cell, which comprises:
a first antigen binding domain which binds a first epitope of the target antigen, a second antigen binding domain which binds a second epitope of the target antigen;
a transmembrane domain; and
an intracellular signalling domain.

The chimeric receptor may be capable of inter-molecular binding, but incapable of intra-molecular binding. In other words the chimeric receptor may be capable of simultaneously binding the first epitope and second epitope of two different target antigen molecules but incapable of simultaneously binding the first epitope and second epitope of the same target antigen molecule. In this way, the chimeric receptor can concatenate target antigen at a T-cell:target cell synapse

The chimeric receptor may comprises first and second polypeptides, in which:
the first polypeptide comprises the first antigen binding domain;
the second polypeptide comprises the second antigen binding domain;
the first and/or second polypeptide comprises a transmembrane domain; and
the first and second polypeptides associate to form the chimeric receptor.

For example the first polypeptide may comprise a heavy chain constant region; and the second polypeptide may comprise a light chain constant region.

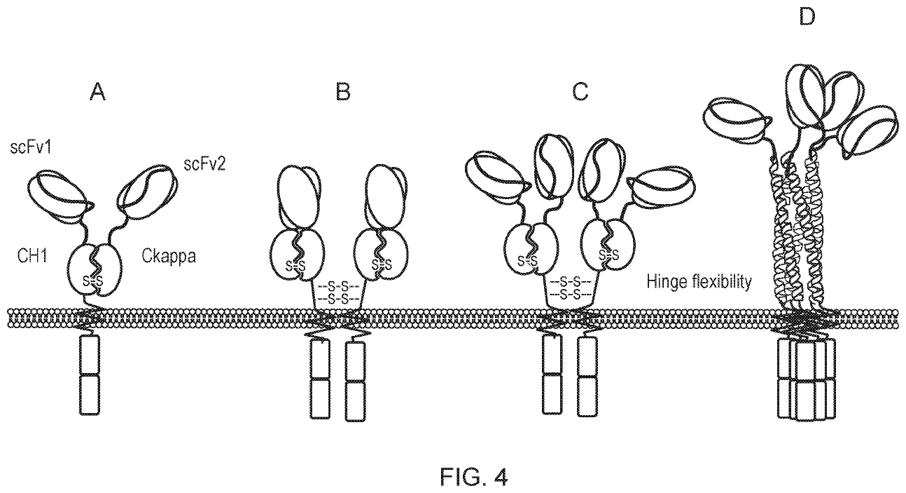

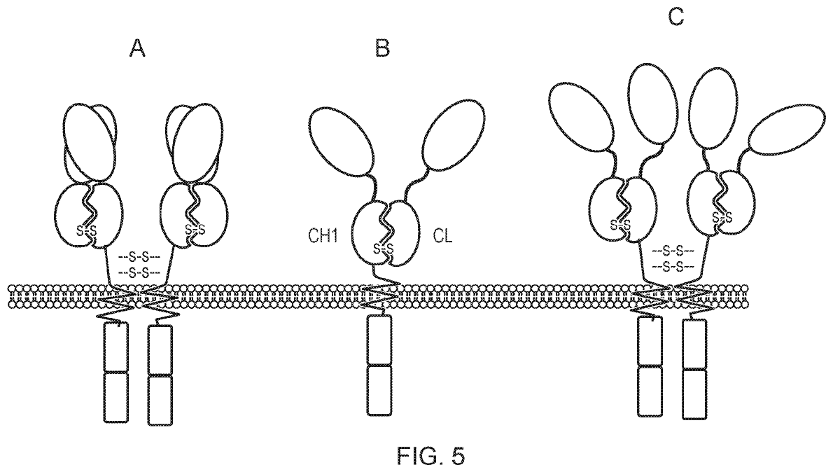

The chimeric receptor may have one of the specific arrangements shown in the Figures, such as: Fab scFv (FIG. 4A); Fab dAb (FIG. 5B); dual Fab scFv (FIG. 4C); dual Fab dAb (FIG. 5C).

The first and second polypeptides may have the general structure:

### ABD-CC-TM

in which ABD is the antigen binding domain, CCS is a coiled-coil spacer domain and TM is a transmembrane domain.

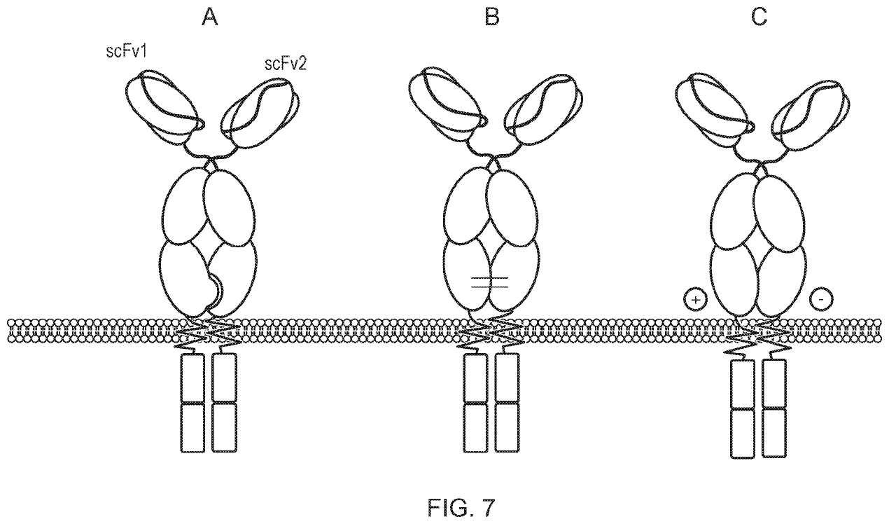

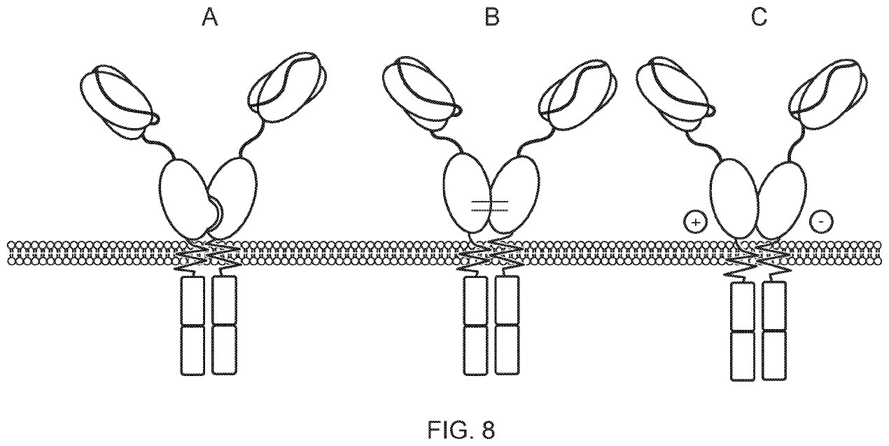

The first and second polypeptides comprise an engineered CH3 domain. For example the chimeric receptor may have one of the structures shown in the Figures, such as: knobs in holes Fc dual scFv (FIG. 7A); strand exchange Fc dual scFv (FIG. 7B); charge pair Fc dual scFv (FIG. 7C); knobs in holes CH3 dual scFv (FIG. 8A); strand exchange CH3 dual scFv (FIG. 8B); charge pair CH3 dual scFv (FIG. 8C); knobs in holes Fc dual dAb; strand exchange Fc dual dAb; charge pair Fc dual dAb; knobs in holes CH3 dual dAb; strand exchange CH3 dual dAb; or charge pair CH3 dual dAb.

The chimeric receptor may comprise two polypeptides, one polypeptide comprising a heavy chain variable region (VH) and the other comprising a light chain variable region (VL) which associate to form the first antigen binding domain.

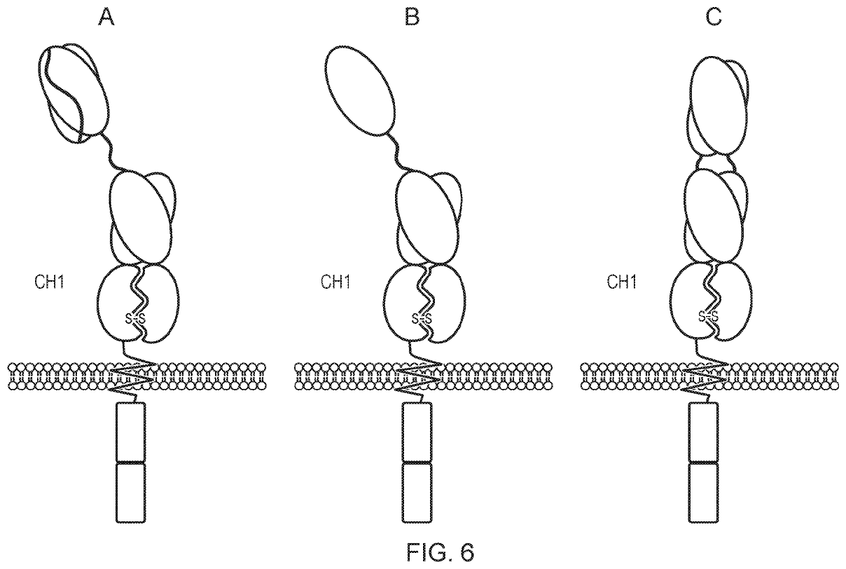

For example, the chimeric receptor may have one of the structures illustrated in the Figures such as: scFv tanFab (FIG. 6A); dAb tanFab (FIG. 6B); and dual variable Fab (FIG. 6C).

The chimeric receptor may comprise four polypeptides:
(i) a first polypeptide which comprises a first heavy chain variable region (VH) and a first heavy chain constant region;
(ii) a second polypeptide which comprises a first light chain variable region (VL) and a first light chain constant region;
(iii) a third polypeptide which comprises a second heavy chain variable region (VH) and a second heavy chain constant region; and
(iv) a fourth polypeptide which comprises a second light chain variable region (VL) and a second light chain constant region;
wherein
the first VH and first VL associate to form the first antigen binding domain;
the second VH and second VL associate to form the second antigen binding domain;
the first and/or second polypeptide chain comprise(s) a transmembrane domain; and
the third and/or fourth polypeptide chain comprise(s) a transmembrane domain

The first VL and the second VL may be the same, but the first VH may be different from the second VH.

The first and second antigen binding domains may be linked on a single polypeptide chain.

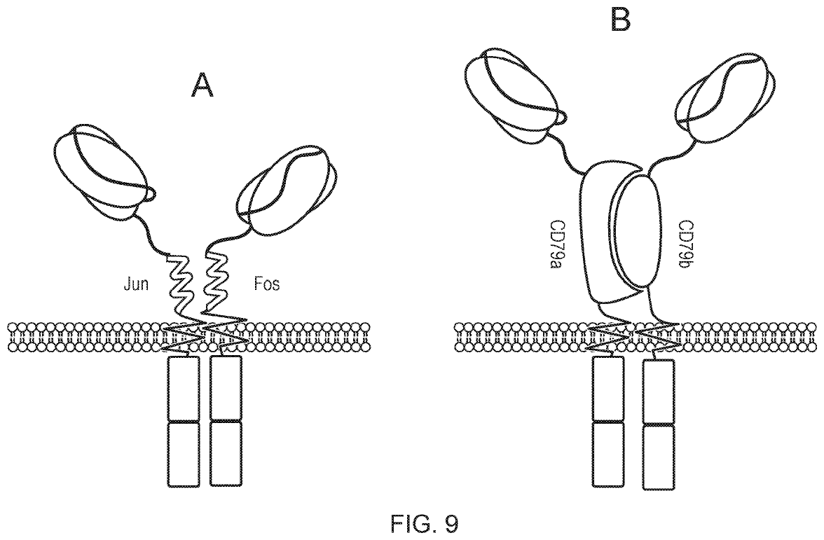

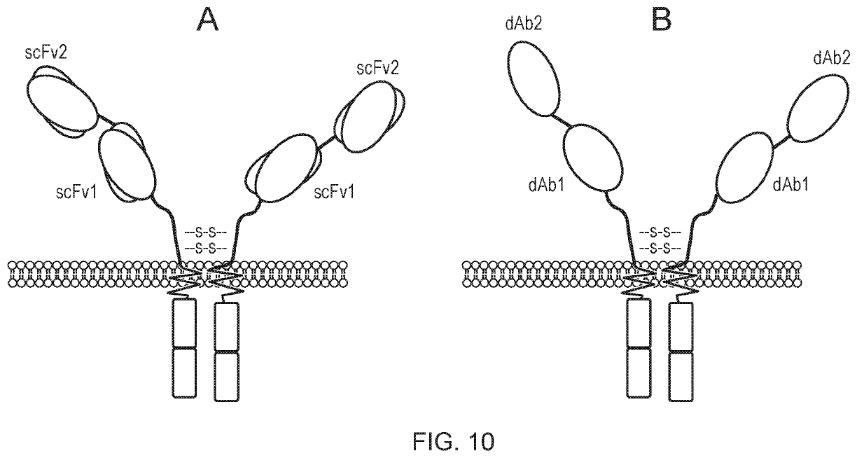

For example, the chimeric receptor may have one of the structures illustrated in the Figures, such as: Leucine zipper Dual-scFv (FIG. 9A); CD79a/b Dual-scFv (FIG. 9B); tanCAR scFv (FIG. 10A); and tanCAR dAb (FIG. 10B).

In any of the embodiments mentioned above, the first epitope may be a membrane proximal epitope and the second epitope may be a membrane distal epitope, or vice versa.

In any of the embodiments mentioned above, the target antigen may be B cell maturation antigen (BCMA), transmembrane activator and calcium modulator and cyclophilin ligand interactor (TACI), CD22 or CD21.

In a second aspect, the present invention provides a cell which comprises a chimeric receptor according to the first aspect of the invention.

In a third aspect, the present invention provides a nucleic acid sequence encoding a chimeric receptor according to the first aspect of the invention.

In a fourth aspect, the present invention provides a nucleic acid construct which comprises: a first nucleic acid sequence encoding a first polypeptide chain as defined in the first aspect of the invention; and a second nucleic acid sequence encoding a second polypeptide chain as defined in the first aspect of the invention.

The nucleic acid construct may comprise: a first nucleic acid sequence encoding a first polypeptide chain as defined in the first aspect of the invention; a second nucleic acid sequence encoding a second polypeptide chain as defined in the first aspect of the invention; a third nucleic acid sequence encoding a third polypeptide chain as defined in the first aspect of the invention; and a fourth nucleic acid sequence encoding a fourth polypeptide chain as defined in the first aspect of the invention.

The nucleic acid construct may comprise: a first nucleic acid sequence encoding a second and fourth polypeptide chain as defined in the first aspect of the invention; a second nucleic acid sequence encoding a first polypeptide chain as defined in the first aspect of the invention; and a third nucleic acid sequence encoding a third polypeptide chain as defined in the first aspect of the invention.

In a fifth aspect there is provided a vector comprising a nucleic acid sequence according to the third aspect of the invention or a nucleic acid construct according to the fourth aspect of the invention.

The vector may, for example, be a retroviral vector, a lentiviral vector or a transposon.

In a sixth aspect, there is provided a kit which comprises:
i) a first vector comprising a nucleic acid sequence encoding a first polypeptide as defined in the first aspect of the invention; and
ii) a second vector comprising a nucleic acid sequence encoding a second polypeptide as defined in the first aspect of the invention.

The kit may comprise:
i) a first vector comprising a nucleic acid sequence encoding a first polypeptide as defined in the first aspect of the invention;
ii) a second vector comprising a nucleic acid sequence encoding a second polypeptide as defined in the first aspect of the invention;
iii) a third vector comprising a nucleic acid sequence encoding a third polypeptide as defined in the first aspect of the invention;
iv) a fourth vector comprising a nucleic acid sequence encoding a fourth polypeptide as defined in the first aspect of the invention.

The kit may comprise:
i) a first vector comprising a first nucleic acid sequence encoding a second and fourth polypeptide chain as defined in the first aspect of the invention;
ii) a second vector comprising a second nucleic acid sequence encoding a first polypeptide chain as defined in the first aspect of the invention; and
iii) a third vector comprising a third nucleic acid sequence encoding a third polypeptide chain as defined in the first aspect of the invention.

In a seventh aspect, there is provided a method for making a cell according to the second aspect of the invention, which comprises the step of introducing: a nucleic acid sequence according to the third aspect of the invention; a nucleic acid construct according to the fourth aspect of the invention; a vector according to the fifth aspect of the invention; or a kit of vectors according to the sixth aspect of the invention, into a cell.

The cell may be from a sample isolated from a subject.

In an eighth aspect, there is provided a pharmaceutical composition comprising a plurality of cells according to the second aspect of the invention.

In a ninth aspect, there is provided a method for treating and/or preventing a disease, which comprises the step of administering a pharmaceutical composition according to the eighth aspect of the invention to a subject.

The method may comprise the following steps:
(i) isolation of a cell-containing sample from a subject;
(ii) transduction or transfection of the cells with: a nucleic acid sequence according to the third aspect of the invention; a nucleic acid construct according to the fourth aspect of the invention; a vector according to the fifth aspect of the invention; or a kit of vectors according to the sixth aspect of the invention; and
(iii) administering the cells from (ii) to a the subject.

The disease may be a cancer.

In a tenth aspect of the invention there is provided a pharmaceutical composition according to the eighth aspect of the invention for use in treating and/or preventing a disease.

In an eleventh aspect there is provided the use of a cell according to the second aspect of the invention in the manufacture of a medicament for treating and/or preventing a disease.

The chimeric receptors of the present invention have two key advantages. Firstly, epitopes that are difficult to access can be targeted by levering down and displacing large target antigens. Secondly, the clustering of CAR and target antigen generates an extensive synapse that is not accessible by inhibitory phosphatases, thereby augmenting CAR-mediated T cell activation.

### DETAILED DESCRIPTION

### Chimeric Receptors

The present invention relates to a chimeric receptor which comprises at least two antigen binding domains.

A classical chimeric antigen receptor (CAR) is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

A CAR may have the general structure:

Antigen binding domain-spacer domain-transmembrane domain-intracellular signaling domain (endodomain).

FabCARs

The chimeric receptor of the present invention may be a FabCAR, which comprises two chains: one having an antibody-like light chain constant region (CL) and one having a heavy chain constant region (CH). Association between the CL and CH causes assembly of the receptor. For all FabCARs mentioned below, there may be a linker between the antigen binding domain (e.g. scFv) or antigen binding domain component (e.g. VH or VL) and the CL or CH domain.

Fab scFv

A Fab scFv chimeric receptor comprises two chains, one with an scFv against a first epitope of the target antigen and one with an scFv against a second epitope of the target antigen (FIG. 4A). The first chain and/or the second chain may comprise a transmembrane domain and an intracellular signalling domain.

The two chains of a Fab scFv may have the general structure:

First scFv-CH-transmembrane domain-intracellular signalling domain; and Second scFv-CL

or

First scFv-CL-transmembrane domain-intracellular signalling domain; and Second scFv-CH

Fab dAb

A Fab dAb chimeric receptor comprises two chains, one with a domain antibody against a first epitope of the target antigen and one with a domain antibody against a second epitope of the target antigen (FIG. 5B). The first chain and/or the second chain may comprise a transmembrane domain and an intracellular signalling domain.

The two chains of a Fab dAb may have the general structure:

First dAb-CH-transmembrane domain-intracellular signalling domain; and Second dAb-CL

or

First dAb-CL-transmembrane domain-intracellular signalling domain; and Second dAb-CH

Dual Fab

The chimeric receptor may be in a dual Fab format (FIG. 4B). In this arrangement, the receptor comprises four polypeptide chains, two of which comprise antibody-like light chain variable domains (VL) and two of which comprise heavy chain variable domains (VH). The receptor comprises two VH:VL pairs forming the two antigen binding domains.

The four chains of a dual Fab chimeric receptor may have the general structure:

First VH-CH-spacer domain-transmembrane domain-intracellular signalling domain;

First VL-CL;

Second VH-CH-spacer domain-transmembrane domain-intracellular signalling domain; and

Second VL-CL

or

First VL-CL-spacer domain-transmembrane domain-intracellular signalling domain;

First VH-CH;

Second VL-CL-spacer domain-transmembrane domain-intracellular signalling domain; and

Second VH-CH

Two of the polypeptide chains in a dual Fab chimeric receptor may be identical. For example, in the arrangement shown in FIG. 5A, the receptor has identical VL domains but different VH domains. This type of molecule may be generated, for example where the antigen binding domains are generated and selected using transgenic animals harbouring human immunoglobulin loci, such as the OmniRat®.

Dual Fab scFv

The chimeric receptor may be in a Dual Fab scFv format, as shown in FIG. 4C. This is similar to the dual Fab arrangement described above, having four chains, but in this arrangement each chain has an scFv-type antigen binding domain. The dual Fab scFv chimeric receptor therefore has two antigen binding domains which bind one target epitope and two antigen binding domains which bind the other target epitope. A dual Fab scFv consist of two pairs of identical chains.

The four chains of a dual Fab scFv may have the general structure:

First scFv-CH-transmembrane domain-spacer domain-intracellular signalling domain;

Second scFv-CL;

First scFv-CH-transmembrane domain-spacer domain-intracellular signalling domain; and

Second scFv-CL

or

First scFv-CL-transmembrane domain-spacer domain-intracellular signalling domain;

Second scFv-CH;

First scFv-CL-transmembrane domain-spacer domain-intracellular signalling domain; and

Second scFv-CH

Dual Fab dAb

The chimeric receptor may be in a Dual Fab dAb format, as shown in FIG. 5C.

The four chains of a Fab dAb may have the general structure:

First dAb-CH-transmembrane domain-spacer domain-intracellular signalling domain;

Second dAb-CL;

First dAb-CH-transmembrane domain-spacer domain-intracellular signalling domain; and

Second dAb-CL

or

First dAb-CL-transmembrane domain-spacer domain-intracellular signalling domain;

Second dAb-CH;

First dAb-CL-transmembrane domain-spacer domain-intracellular signalling domain; and

Second dAb-CH.

ScFv tan Fab

An scFv tanFab chimeric receptor (FIG. 6A) comprises two polypeptide chains, one which provides an scFv which binds the first epitope of the target antigen together with either the VH or VL of the antigen binding domain which binds the second epitope of the target antigen. The other polypeptide chain provides the other part of the VH:VL which binds the second epitope of the target antigen. The first chain and/or the second chain may comprise a transmembrane domain and an intracellular signalling domain.

The two chains of an scFv tanFab may have the general structure:

ScFv-VH-CH-transmembrane domain-intracellular signalling domain; and VL-CL

or

ScFv-VL-CL-transmembrane domain-intracellular signalling domain; and VH-CH

The scFv element can alternatively be placed on a polypeptide chain without a transmembrane chain, i.e.

VH-CH-transmembrane domain-intracellular signalling domain; and

scFv-VL-CL

or

VL-CL-transmembrane domain-intracellular signalling domain; and scFv-VH-CH

dAb tan Fab

A dAb tanFab chimeric receptor (FIG. 6B) comprises two polypeptide chains, one which provides an domain binder which binds the first epitope of the target antigen together with either the VH or VL of the antigen binding domain which binds the second epitope of the target antigen. The other polypeptide chain provides the other part of the VH:VL which binds the second epitope of the target antigen. The first chain and/or the second chain may comprise a transmembrane domain and an intracellular signalling domain.

The two chains of a dAb tanFab may have the general structure:

dAb-VH-CH-transmembrane domain-intracellular signalling domain; and

VL-CL

or

dAb-VL-CL-transmembrane domain-intracellular signalling domain; and

VH-CH

The scFv element can alternatively be placed on a polypeptide chain without a transmembrane chain, i.e.

VH-CH-transmembrane domain-intracellular signalling domain; and dAb-VL-CL

or

VL-CL-transmembrane domain-intracellular signalling domain; and dAb-VH-CH.

Dual Variable Fab

A dual variable Fab chimeric receptor (FIG. 6C) comprises two polypeptide chains, one which provides either the VH or VL of the antigen binding domain which binds the first epitope of the target antigen and either the VH or VL of the antigen binding domain which binds the second epitope of the target antigen. The other polypeptide chain provides the other part of the VH:VL which binds the first and second epitopes of the target antigen. The first chain and/or the second chain may comprise a transmembrane domain and an intracellular signalling domain.

The two chains of a dual variable Fab may have the general structure:

VH1-VH2-CH-transmembrane domain-intracellular signalling domain; and

VL1-VL2-CL

or

VL1-VL2-CL-transmembrane domain-intracellular signalling domain; and VH1-VH2-CH

The VL and VH domains may alternatively be mixed on both chains, for example:

VH1-VL2-CH-transmembrane domain-intracellular signalling domain; and VL1-VH2-CL

or

VL1-VH2-CL-transmembrane domain-intracellular signalling domain; and

VH1-VL2-CH

Fc and CH3 Chimeric Receptors

The chimeric receptor of the present invention may comprise Fc-type domains, i.e. CH2-CH3 domains. In this embodiment, the chimeric receptor comprises two chains, in which one polypeptide provides the first antigen binding domain and the second polypeptide provides the second antigen binding domain. Both polypeptides have an Fc domain. Association between the two Fc domains causes assembly of the receptor. For all Fc and CH3 chimeric receptors mentioned below, there may be a linker between the antigen binding domain (e.g. scFv) or antigen binding domain component (e.g. VH or VL) and the Fc or CH3 domain.

The two chains of an Fc dual scFv may have the general structure:

ScFv1-Fc-transmembrane domain-intracellular signalling domain; and

ScFv2-Fc-transmembrane domain-intracellular signalling domain

The two chains of an Fc dual dAb may have the general structure:

dAb1-Fc-transmembrane domain-intracellular signalling domain; and

dAb2-Fc-transmembrane domain-intracellular signalling domain

The transmembrane and/or intracellular signalling domains of the two chains may be the same or different. Alternatively, one chain may lack a transmembrane domains and/or an intracellular signalling domain.

The chimeric receptor of the present invention may comprise Fc-type CH3 domains. In this embodiment, the chimeric receptor comprises two chains, in which one polypeptide provides the first antigen binding domain and the second polypeptide provides the second antigen binding domain. Both polypeptides have a CH3 domain. Association between the two CH3 domains causes assembly of the receptor.

The two chains of a CH3 dual scFv may have the general structure:

ScFv1-CH3-transmembrane domain-intracellular signalling domain; and

ScFv2-CH3-transmembrane domain-intracellular signalling domain

The two chains of an CH3 dual dAb may have the general structure:

dAb1-CH3-transmembrane domain-intracellular signalling domain; and

dAb2-CH3-transmembrane domain-intracellular signalling domain

The transmembrane and/or intracellular signalling domains of the two chains may be the same or different. Alternatively, one chain may lack a transmembrane domains and/or an intracellular signalling domain.

The Fc or CH3 parts of the chimeric receptor may be modified to strengthen the association between the two domains.

For example, “knobs-into-holes” antibody engineering has been described in which one chain is modified to be the “knob” variant by replacement of a small amino acid with a larger one in the CH3 domain; and the other chain is modified to be the “hole” by replacement of a large amino acid with a smaller one. For example a T366Y mutation may be used to create the knob variant and a Y407T mutation may be used to create the hole variant. This technology has been previously described for producing bifunctional antibodies, but can be equally applied to the chimeric receptors of the present invention.

A pair of knobs-into-holes Fc sequences are shown below as SEQ ID Nos. 6 and 7.

The strand-exchange engineered domain (SEED) platform has also been described for generating asymmetric and bispecific antibody-like molecules. This protein engineered platform is based on exchanging structurally related sequences within the CH3 domains. Alternating sequences from human IgA and IgG in the SEED CH3 domains generate two asymmetric but complementary domains, designated AG and GA. The SEED design allows efficient generation of AG/GA heterodimers, while disfavoring homodimerization of AG and GA SEED CH3 domains.

A pair of strand exchange Fc sequences are shown below as SEQ ID Nos. 8 and 9.

Fc interaction can also be enhanced by modifying the CH3 domain interface of the antibody Fc region with selected mutations so that the engineered Fc proteins preferentially form heterodimers. These novel mutations create altered charge polarity across the Fc dimer interface such that coexpression of electrostatically matched Fc chains support favorable attractive interactions thereby promoting desired Fc heterodimer formation, whereas unfavorable repulsive charge interactions suppress unwanted Fc homodimer formation. Due to the 2-fold symmetry of the Fc, each unique interaction at the CH3-CH3 domain interface is represented twice in the structure. The electrostatic steering mechanism exploits the same 2-fold symmetry to effectively hinder the homodimer formation. A single mutation such as K409D in the first chain or D399′K in the second chain makes use of the symmetry to impart a repulsive electrostatic interaction in the homodimer setting. This repulsive effect can be further enhanced by combining different charge mutations, for example K409D: K392 D: K370D and D399′K:E356′K:E357′K.

A pair of charge pair Fc sequences are shown below as SEQ ID Nos. 10 and 11.

CD79a/b Chimeric Receptors

CD79 is a transmembrane protein that forms a complex with the B-cell receptor (BCR) and generates a signal following recognition of antigen by the BCR. CD79 is composed of two distinct chains: CD79a (Uniprot: P11912) and CD79b (Uniprot: P40259) which form a heterodimer on the surface of a B cell stabilized by disulfide bonding.

The chimeric receptor of the present invention may comprise the ectodomains of CD79a and CD79b. In this embodiment, the chimeric receptor comprises two chains, in which one polypeptide provides the first antigen binding domain and the second polypeptide provides the second antigen binding domain. One polypeptide comprises the CD79a domain and one polypeptide comprises the CD79b domain. Association between the two CD79 domains causes assembly of the receptor. For CD79a/b chimeric receptors described below, there may be a linker between the antigen binding domain (e.g. scFv) or antigen binding domain component (e.g. VH or VL) and the CD79a or CD79b domain.

The two chains of an CD79a/b dual scFv may have the general structure:

ScFv1-CD79a-transmembrane domain-intracellular signalling domain; and

ScFv2-CD79b-transmembrane domain-intracellular signalling domain

The two chains of an Fc dual dAb may have the general structure:

dAb1-CD79a-transmembrane domain-intracellular signalling domain; and

dAb2-CD79b-transmembrane domain-intracellular signalling domain

The transmembrane and/or intracellular signalling domains of the two chains may be the same or different. Alternatively, one chain may lack a transmembrane domains and/or an intracellular signalling domain.

Suitable CD79a and CD79b ectodomain sequences for use in the chimeric receptor of the present invention are shown below as SEQ ID Nos 12 and 13.

Leucine Zipper Chimeric Receptors

The chimeric receptor of the present invention may comprise a pari of domains which spontaneously heterodimerise, such as a leucine zipper. Leucine zippers and other heretodimerising domain pairs such as DDD1 and AD1 domains, Barnase and Barnstar domains or human pancreatic RNAse and S-peptide domains, are described in WO2016/124930.

The leucine zipper is a super-secondary structure that functions as a dimerization domain. Its presence generates adhesion forces in parallel alpha helices. A single leucine zipper consists of multiple leucine residues at approximately 7-residue intervals, which forms an amphipathic alpha helix with a hydrophobic region running along one side. This hydrophobic region provides an area for dimerization, allowing the motifs to “zip” together. Leucine zippers are typically 20 to 40 amino acids in length, for example approximately 30 amino acids.

In this embodiment of the present invention, the chimeric receptor comprises two chains, in which one polypeptide provides the first antigen binding domain and the second polypeptide provides the second antigen binding domain. One polypeptide comprises, for example, a Jun leucine zipper domain and one polypeptide comprises a Fos leucine zipper domain. Association between the Jun and Fos domains causes assembly of the receptor. For the leucine zipper chimeric receptors described below, there may be a linker between the antigen binding domain (e.g. scFv) or antigen binding domain component (e.g. VH or VL) and the leucine zipper domain.

The two chains of a leucine zipper dual scFv may have the general structure:

ScFv1-Jun-transmembrane domain-intracellular signalling domain; and

ScFv2-Fos-transmembrane domain-intracellular signalling domain

The two chains of a leucine zipper dual dAb may have the general structure:

dAb1-Jun-transmembrane domain-intracellular signalling domain; and

dAb2-Fos-transmembrane domain-intracellular signalling domain

The transmembrane and/or intracellular signalling domains of the two chains may be the same or different. Alternatively, one chain may lack a transmembrane domains and/or an intracellular signalling domain.

Suitable Fos and Jun leucine zipper domain sequences for use in the chimeric receptor of the present invention are shown below as SEQ ID Nos 14 and 15.

TanCARs

The chimeric receptor may be a “tandem CAR” or “tanCAR”. These receptors are based on the design of a classical CAR, as described above, but are bi-specific, having two antigen-binding domains connected by a linker. The antigen binding domains may, for example be single-chain variable fragments (scFvs) or single domain antibodies (dAbs). Grada et al (2013, Molecular Therapy 2:e105) describes a tanCAR targeting CD19 and human epidermal growth factor receptor 2. In a tanCAR of the present invention, the two binding domains target different epitopes of the same target antigen. The linker may be designed to give optimal spatial positioning of the two antigen binding domains to target the two separate epitopes on neighbouring target antigen molecules.

A tanCAR may have the general structure:

First antigen binding domain-linker-second antigen binding domain-spacer domain-transmembrane domain-intracellular signalling domain.

Antigen Binding Domain

The antigen binding domain is the portion of the chimeric receptor which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a single domain antibody (dAb); an artificial single binder such as a Darpin (designed ankyrin repeat protein); a single-chain derived from a T-cell receptor; a natural ligand of the target antigen; or a peptide with sufficient affinity for the target.

For the Fab-type chimeric receptors described above, the antigen binding domain may be an scFv or may be made up of a VH from one polypeptide chain and a VL from another polypeptide chain.

In the chimeric receptor of the present invention the two (or more) antigen binding domains bind to mutually exclusive epitopes of the target antigen. The epitopes may, for example, be non-overlapping. The first and second antigen binding domains do not compete with each other for binding to the first or second epitope. The capacity of two antigen binding domains to bind to two epitopes of a target antigen without competing with each other may readily be determined using a competition assay.

The two target epitopes may be located in different domains of the target antigen. For example, in the case of CD22 which comprises seven Ig-like domains, the first and second epitopes may be located on different Ig-like domains.

One target epitope may be located in a membrane distal position on the target antigen and the other target epitope may be located in a membrane proximal position on the target antigen. For long target antigens, binding to the membrane proximal epitope may “bend” the antigen, making the membrane distal epitope easier to access for the chimeric receptor. Binding of both the membrane distal and membrane proximal epitope may have the effect of flattening a long target antigen, which can result in a better T-cell: target cell synapse.

The first antigen binding domain and second antigen binding domain may not be capable of intra-molecular binding, i.e. they may not be capable of simultaneously binding the first and second epitopes of an individual target antigen molecule.

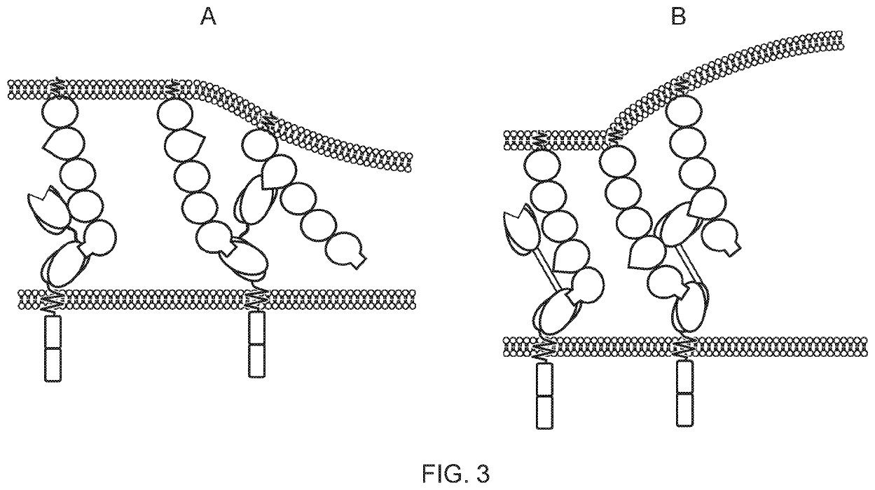

This may be because the distance between the two epitopes on the target antigen is such that it is spatially impossible for the two antigen binding domains to “reach” both epitopes simultaneously. The spacer of the chimeric receptor and/or any linker between the antigen binding domain or VL/VH and the spacer/CUCH can be designed and selected so as to prevent intra-molecular binding. For example, as shown in FIG. 3A, where the two epitopes are spatially separated on the target antigen, the use of a short, flexible linker can be used to prevent intra-molecular binding but allow inter-molecular binding. Where the two epitopes are located relatively near to each other on the target antigen, a longer but more rigid linker may be used prevent intra-molecular binding but allow inter-molecular binding (FIG. 3B).

Linker

For the Fab-type, Fv and CH3 chimeric receptors described above, the polypeptide chains may comprise a linker between the scFv or VH/VL domain and the CH/CL, Fc or CH3 domain. The linker may be the same or different in the two (or four) polypeptide chains.

The linker may be flexible and serve to spatially separate the scFv or VH/VL domain from the CH/CL, Fc or CH3 domain.

Flexible linkers may be composed of small, non-polar residues such as glycine, threonine and serine. The linker may comprise one or more repeats of a glycine-serine linker, such as a (Gly₄Ser)ₙ linker, where n is the number of repeats.

The or each linker may be less than 50, 40, 30, 20 or 10 amino acids in length. The or each linker may be selected to give optimal spatial positioning for the first and second antigen-binding domains to bind the first and second epitopes of the target antigen on neighbouring target antigen molecules.

A rigid linker may, for example, be a helical linker such as (EAAAK)n where n>4. This linker spans a maximum distance of 12 nm when n=4.

A chimeric receptor with an scFv antigen binding domain may include a linker such as the one shown in SEQ ID No. 62

SEQ ID No. 62
DPAEPKSPDKTHTCPPCP

Where a chimeric receptor have two polypeptides, one contributing a VL domain and one contributing a VH domain, one of the following linkers may be used

(VH linker long)
SEQ ID No. 63
ASTKGPSVFPLAP
(VL linker long)
SEQ ID NO. 64
TVAAPSVFIFPP
(VH linker short)
SEQ ID NO. 65
ASTKGP
(VL linker short)
SEQ ID NO. 66
TVAAP

Spacer

Classical CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

In the Fab-type chimeric receptors discussed above, the “spacer” comprises a CH or CL domain.

There are two types of light chain in humans: kappa (κ) chain and lambda (λ) chain. The lambda class has 4 subtypes: λ₁, λ₂, λ₃ and λ₄. The light chain constant region of a Fab-type chimeric receptor may be derived from any of these light chain types.

The light chain constant domain of a chimeric receptor of the present invention may have the sequence shown as SEQ ID NO. 1 which is a kappa chain constant domain.

SEQ ID No. 1
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

There are five types of mammalian immunoglobulin heavy chain: γ, δ, α, μ and ε which define the classes of immunoglobulin IgG, IgD, IgA, IgM and IgE respectively.

Heavy chains γ, δ and α have a constant domain composed of three tandem Ig domain and have a hinge for added flexibility. Heavy chains μ and ε are composed of four domains.

The CH domain of a Fab-type chimeric receptor of the present invention may comprise the sequence shown as SEQ ID No. 2 which is from a γ immunoglobulin heavy chain.

SEQ ID No. 2
STKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV

In a dual FAB and dual Fab scFv format (FIGS. 4B and C), as in a classical chimeric antigen receptor, the spacer may cause two of the polypeptide chains to dimerise. Two of the polypeptide chains may, for example, comprise one or more suitable cysteine residues to form di-sulphide bridge(s). The hinge from IgG1 is suitable in this regard. A spacer based on an IgG1 hinge may have the sequence shown as SEQ ID. No. 3

(human IgG1 hinge):
SEQ ID No. 3
AEPKSPDKTHTCPPCPKDPK

Alternatively, a hinge spacer may have the sequence shown as SEQ ID No. 17

(hinge spacer)
SEQ ID No. 17
EPKSCDKTHTCPPCP

For Fc and CH3 chimeric receptors mentioned above, the spacer is an antibody-like Fc domain or a CH3 domain respectively.

The wild-type sequence of IgG-derived Fc and CH3 are shown as SEQ ID Nos 4 and 5 below.

(Heavy chain CH2CH3)
SEQ ID No. 4
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK
(Heavy chain CH3)
SEQ ID No. 5
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The Fc or CH3 parts of the chimeric receptor may be modified to strengthen the association between the two domains, using for example “knob-into-holes” technology, strand exchange or electrostatic steering, as described above.

A pair of knobs-into-holes Fc sequences are shown below as SEQ ID Nos. 6 and 7 Mutated residues are shown in bold.

(HCH2CH3pvaa_KIHa)
SEQ ID No. 6
APPVAGPSVFLFPPKPKDTLMIARTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVCTLPPSRDELTKNQVSLSCAVKGFYPSDIAVEW
ESNGQPENNYKTTPPVLDSDGSFFLVSKLTVDKSRWQQGNVFSCSVMHEA
LHNHYTQKSLSLSPGK
(HCH2CH3pvaa_KIHb)
SEQ ID No. 7
APPVAGPSVFLFPPKPKDTLMIARTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPCRDELTKNQVSLWCLVKGFYPSDIAVEW
ESNGQPENNYKTTPPVLDSDGSFFLYSKLTVDKSRWQQGNVFSCSVMHEA
LHNHYTQKSLSLSPGK

A pair of strand exchange Fc sequences are shown below as SEQ ID Nos. 8 and 9 Mutated residues are shown in bold.

(HCH2CH3pvaa_StrandExa)
SEQ ID No. 8
APPVAGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPPSEELALNELVTLTCLVKGFYPSDIAVE
WLQGSQELPREKYLTWAPVLDSDGSFFLYSILRVAAEDWKKGDTFSCSVM
HEALHNHYTQKSLDRSPGK
(HCH2CH3pvaa_StrandExb)
SEQ ID No. 9
APPVAGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAP
IEKTISKAKGQPFRPEVHLLPPSREEMTKNQVSLTCLARGFYPKDIAVEW
ESNGQPENNYKTTPSRQEPSQGTTTFAVTSKLTVDKSRWQQGNVFSCSVM
HEALHNHYTQKTISLSPGK

A pair of charge pair Fc sequences are shown below as SEQ ID Nos. 10 and 11 Mutated residues are shown in bold.

(HCH2CH3pvaa_ESa)
SEQ ID No. 10
APPVAGPSVFLFPPKPKDTLMIARTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEW
ESNGQPENNYDTTPPVLDSDGSFFLYSDLTVDKSRWQQGNVFSCSVMHEA
LHNHYTQKSLSLSPGK
(HCH2CH3pvaa_ESb)
SEQ ID No. 11
APPVAGPSVFLFPPKPKDTLMIARTPEVTCVVVDVSHEDPEVKFNWYVDG
VEVHNAKTKPREEQYNSTYRVVSVLTVLHQDWLNGKEYKCKVSNKALPAP
IEKTISKAKGQPREPQVYTLPPSRDKLTKNQVSLTCLVKGFYPSDIAVEW
ESNGQPENNYKTTPPVLKSDGSFFLYSKLTVDKSRWQQGNVFSCSVMHEA
LHNHYTQKSLSLSPGK

For CD79a/b chimeric receptors, the spacer on one polypeptide is the CD79a ectodomain and the spacer on the other polypeptide is the CD79b ectodomain. Suitable sequences are shown as SEQ ID Nos. 12 and 13 below.

(CD79a ectodomain)
SEQ ID No. 12
LWMHKVPASLMVSLGEDAHFQCPHNSSNNANVTVWVRVLHGNYTWPPEFL
GPGEDPNGTLIIQNVNKSHGGIYVCRVQEGNESYQQSCGTYLRVRQPPPR
PFLDMGEGTKNR
(CD79b ectodomain)
SEQ ID No. 13
ARSEDRYRNPKGSACSRIWQSPRFIARKRGFTVKMHCYMNSASGNVSWLW
KQEMDENPQQLKLEKGRMEESQNESLATLTIQGIRFEDNGIYFCQQKCNN
TSEVYQGCGTELRVMGFSTLAQLKQRNTLKD

For leucine zipper chimeric receptors, the spacer on one polypeptide is the Fos leucine zipper domain and the spacer on the other polypeptide is the Jun leucine zipper domain. Suitable sequences are shown as SEQ ID Nos. 14 and 15 below.

(Fos-Leucine zipper)
SEQ ID No. 14
LTATLQAETDQLEDEKSALQTEIANLLKEKEKLEFILAA
(Jun-Leucine zipper)
SEQ ID No. 15
LEEKVKTLKAQNSELASTANMLREQVAQLKQKVMN

For tanCARs and Fab tanCARs the spacer may be any sequence which spatially separates the antigen binding domains from the transmembrane domains, or from the VH/VL domains of a Fab-based antigen binding domain, allowing the antigen-binding domain(s) to have suitable orientation and reach.

Commonly used CAR spacers include a human an IgG1 Fc domain; an IgG1 hinge; an IgG1 hinge-CD8 stalk; or a CD8 stalk.

In an alternative embodiment of the present invention, the chimeric receptor may comprise a coiled-coil spacer domain (FIG. 4D). Chimeric antigen receptors having coiled-coil spacer domains are described in WO2016/151315.

A coiled coil is a structural motif in which two to seven alpha-helices are wrapped together like the strands of a rope. Many endogenous proteins incorporate coiled coil domains.

Coiled coils usually contain a repeated pattern, hxxhcxc, of hydrophobic (h) and charged (c) amino-acid residues, referred to as a heptad repeat. The positions in the heptad repeat are usually labeled abcdefg, where a and d are the hydrophobic positions, often being occupied by isoleucine, leucine, or valine. Folding a sequence with this repeating pattern into an alpha-helical secondary structure causes the hydrophobic residues to be presented as a ‘stripe’ that coils gently around the helix in left-handed fashion, forming an amphipathic structure. The most favourable way for two such helices to arrange themselves in the cytoplasm is to wrap the hydrophobic strands against each other sandwiched between the hydrophilic amino acids. Thus, it is the burial of hydrophobic surfaces that provides the thermodynamic driving force for the oligomerization. The packing in a coiled-coil interface is exceptionally tight, with almost complete van der Waals contact between the side-chains of the a and d residues.

Examples of coiled coil domains which are capable of forming multimers comprising more than two coiled coil domains include, but are not limited to, those from cartilage-oligomeric matrix protein (COMP), mannose-binding protein A, coiled-coil serine-rich protein 1, polypeptide release factor 2, SNAP-25, SNARE, Lac repressor or apolipoprotein E.

The coiled coil domain may be a COMP coiled coil domain which forms a pentamer.

The coiled coil domain may consist of or comprise the sequence shown as SEQ ID No. 16 or a fragment thereof.

SEQ ID No. 16
DLGPQMLRELQETNAALQDVRELLRQQVREITFLKNTVMECDACG

It is possible to truncate the COMP coiled-coil domain at the N-terminus and retain surface expression. The coiled-coil domain may therefore comprise or consist of a truncated version of SEQ ID No. 16, which is truncated at the N-terminus. The truncated COMP may comprise the 5 C-terminal amino acids of SEQ ID No. 16, i.e. the sequence CDACG. The truncated COMP may comprise 5 to 44 amino acids, for example, at least 5, 10, 15, 20, 25, 30, 35 or 40 amino acids. The truncated COMP may correspond to the C-terminus of SEQ ID No. 16. For example a truncated COMP comprising 20 amino acids may comprise the sequences QQVREITFLKNTVMECDACG. Truncated COMP may retain the cysteine residue(s) involved in multimerisation. Truncated COMP may retain the capacity to form multimers.

Various coiled coil domains are known which form hexamers such as gp41 derived from HIV, and an artificial protein designed hexamer coiled coil described by N. Zaccai et al. (2011) Nature Chem. Bio., (7) 935-941). A mutant form of the GCN4-p1 leucine zipper forms a heptameric coiled-coil structure (J. Liu. et al., (2006) PNAS (103) 15457-15462).

Transmembrane Domain

The transmembrane domain is the portion of the chimeric receptor which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the chimeric receptor. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

The transmembrane domain may be derived from CD28, which gives good receptor stability.

Endodomain

The endodomain is the signal-transmission portion of the chimeric receptor. It may be part of or associate with the intracellular domain of the chimeric receptor. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:

(i) an ITAM-containing endodomain, such as the endodomain from CD3 zeta; and/or

(ii) a co-stimulatory domain, such as the endodomain from CD28 or ICOS; and/or

(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The chimeric receptor of the present invention may therefore comprise an antigen-binding component comprising an antigen-binding domain and a transmembrane domain; which is capable of interacting with a separate intracellular signalling component comprising a signalling domain. The vector of the invention may express a chimeric receptor signalling system comprising such an antigen-binding component and intracellular signalling component.

The chimeric receptor may comprise a signal peptide so that when it is expressed inside a cell, the nascent protein is directed to the endoplasmic reticulum and subsequently to the cell surface, where it is expressed. The signal peptide may be at the amino terminus of the molecule.

Target Antigen

A ‘target antigen’ is an entity which is specifically recognised and bound by the antigen-binding domains of a chimeric receptor of the invention.

The target antigen may be an antigen present on a cancer cell, for example a tumour-associated antigen.

The target antigen for the chimeric receptor may be expressed at relatively low density on the target cell.

The cells of the present invention may be capable of killing target cells, such as cancer cells, which express a low density of the CAR target antigen. Examples of tumour associated antigens which are known to be expressed at low densities in certain cancers include, but are not limited to, ROR1 in CLL, Typr-1 in melanoma, BCMA and TACI in myeloma, CD22 in B-cell malignancies and ALK in Neuroblastoma.

The mean copy number of the target antigen may be fewer than about 10,000; 5,000; 3,000; 2,000; 1,000; or 500 copies per target cell.

The copy number of an antigen on a cell, such as a cancer cell may be measured using standard techniques, such as using PE Quantibrite beads.

The target antigen may have a relatively long and/or bulky extracellular domain. The extracellular domain of CD22 has seven IgG-like domains in its extracellular domain. The target antigen of the chimeric receptor of the invention may have a length equivalent to at least 4, 5, 6 or 7 Ig-like domains. The extracellular domain of CD21 has 21 short consensus repeats (SCR) of about 60 amino acids each. The target antigen of the chimeric receptor of the invention may have a length equivalent to at least 15, 17, 19 or 21 CSRs.

The target antigen may have an extracellular domain which is longer than the optimal intracellular distance between a T-cell and a target cell at a T-cell:target cell synapse. The target cell may have an extracellular domain which is at least 40, 50, 60 or 70 nM

The target antigen may be CD22, CD21, BCMA or TACI

CD22

CD22 has seven extracellular IgG-like domains, which are commonly identified as Ig domain 1 to Ig domain 7, with Ig domain 7 being most proximal to the B cell membrane and Ig domain 1 being the most distal from the Ig cell membrane.

The positions of the Ig domains in terms of the amino acid sequence of CD22 (http://www.uniprot.org/uniprot/P20273) are summarised in the following table:

| Ig | Amino |
|---|---|
| domain | acids |
| 7 | 20-138 |
| 6 | 143-235 |
| 5 | 242-326 |
| 4 | 331-416 |
| 3 | 419-500 |
| 2 | 505-582 |
| 1 | 593-676 |

Examples of anti-CD22 CARs with antigen-binding domains derived from m971, HA22 and BL22 scFvs are described by Haso et al. (Blood; 2013; 121(7)). The antibodies HA22 and BL22 bind to an epitope on Ig domain 5 of CD22.

Other anti-CD22 antibodies are known, such as the mouse anti-human CD22 antibodies 1D9-3, 3B4-13, 7G6-6, 6C4-6, 4D9-12, 5H4-9, 10C1-D9, 15G7-2, 2B12-8, 2C4-4 and 3E10-7; and the humanised anti-human CD22 antibodies LT22 and Inotuzumab (G5_44). The present application describes new VHH-type single domain binders A7 and B4. Table 1 summarises the, VH, VL and CDR sequences (in bold and underlined) and the position of the target epitope on CD22 for each antibody, and the VHH and CDR sequence for each VHH binder.

A number of definitions of the CDRs are commonly in use. The Kabat definition is based on sequence variability and is the most commonly used (see http://www.bioinf.org.uk/abs/). The ImMunoGeneTics information system (IMGT) (see http://www.imgt.org) can also be used. According to this system, a complementarity determining region (CDR-IMGT) is a loop region of a variable domain, delimited according to the IMGT unique numbering for V domain. There are three CDR-IMGT in a variable domain: CDR1-IMGT (loop BC), CDR2-IMGT (loop C′C″), and CDR3-IMGT (loop FG). Other definitions of the CDRs have also been developed, such as the Chothia, the AbM and the contact definitions (see http://www.imgt.org). In Table 1, the sequences are labelled as “Kabat” or “IMGT” depending on which system was used to derive the CDRs.

TABLE 1

| | | | Position |
|---|---|---|---|
| | | | of epitope |
| Antibody | VH | VL | on CD22 |
| 1D9-3 | EVQLVESGGGLVQPKGSLKLSCAAS | DIVMTQSQKFMSTSVGDRVSITC | Domain 5 |
| | GFTFNTYAMHWVRQAPGKGLEWVAR | KASQNVRTAVAWYQQKPGQSPKA | |
| | IRSKSSNYATYYADSVKDRFTISRD | LIYLASNRHTGVPDRFTGSGSGT | |
| | DSQSMLYLQMNNLKTEDTAMYYCVV | DFTLTISNVQSEDLADYFCLQHW | |
| | DYLYAMDYWGQGTSVTVSS | NYPFTFGSGTKLEIKR | |
| | (SEQ ID No. 18) [IMGT] | (SEQ ID No. 19) [INGT] | |
| 3B4-13 | QVQLQQSGAELVRPGASVTLSCKAS | QAVVTQESALTTSPGETVTLTCR | Domain 1 |
| | GYTFTDYEMHWVKQTPVHGLEWIGA | SSAGAVTTSNYANWVQEKPDHLF | and 2 |
| | IDPETGATAYNQKFKGKAILTADKS | TGLIGGTNNRAPGVPARFSGSLI | |
| | SSTAYMDLRSLTSEDSAVYYCTRYD | GDKAALTITGAQTEDEAIYFCAL | |
| | YGSSPWFAYWGQGTLVTVSA | WNSNHWVFGGGTKLTVL | |
| | (SEQ ID No. 20) [Kabat] | (SEQ ID No. 21) [Kabat] | |
| 7G6-6 | QVQLQQPGAELVMPGASVKLSCKAS | DIVMSQSPSSLAVSVGEKVTMSC | Domain 1 |
| | GYTFTSYWMHWVKQRPGQGLEWIGE | KSSQSLLYSSNQKNYLAWYQQKP | and 2 |
| | IDPSDSYTNYNQKFKGKATLTVDKS | GQSPKLLIYWASTRESGVPDRFT | |
| | SSTAYMQLSSLTSEDSAVYYCARGY | GSGSGTDFTLTISSVKAEDLAVY | |
| | YGSSSFDYWGQGTTLTVSS | YCQQYYSYTFGGGTKLEIK | |
| | (SEQ ID No. 22) [Kabat] | (SEQ ID No. 23) [Kabat] | |
| 6C4-6 | QVQLKESGPGLVAPSQSLSITCTVS | DIQMTQSPASLSASVGETVTITC | Domain 3 |
| | GFSLTSYGVHWVRQPPGKGLEWLVV | RASENIYSYLAWYQQKQGKSPQL | |
| | IWSDGSTTYNSALKSRLSISKDNSK | LVYNAKTLAEGVPSRFSGSGSGT | |
| | SQVFLKMNSLQTDDTAMYYCARHAD | QFSLKINSLQPEDFGSYYCQHHY | |
| | DYGFAWFAYWGQGTLVTVSA | GTPPTFGGGTKLEIK | |
| | (SEQ ID No. 24) [Kabat] | (SEQ ID No. 25) [Kabat] | |
| 4D9-12 | EFQLQQSGPELVKPGASVKISCKAS | DIQMTQSPSSLSASLGERVSLTC | Domain 4 |
| | GYSFTDYNMNWVKQSNGKSLEWIGV | RASQEISGYLSWLQQKPDGTIKR | |
| | INPNYGTTSYNQKFKGKATLTVDQS | LIYAASTLDSGVPKRFSGSRSGS | |
| | SSTAYMQLNSLTSEDSAVYYCARSS | DYSLTISSLESEDFADYYCLQYA | |
| | TTVVDWYFDVWGTGTTVTVSS | SYPFTFGSGTKLEIK | |
| | (SEQ ID No. 26) [Kabat] | (SEQ ID No. 27) [Kabat] | |
| 5H4-9 | QVQVQQPGAELVRPGTSVKLSCKAS | DVVMTQTPLSLPVSLGDQASISC | Domain 4 |
| | GYTFTRYWMYWVKQRPGQGLEWIGV | RSSQSLVHSNGNTYLHWYLQKPG | |
| | IDPSDNFTYYNQKFKGKATLTVDTS | QSPKLLIYKVSNRFSGVPDRFSG | |
| | SSTAYMQLSSLTSEDSAVYYCARGY | SGSGTDFTLKISRVEAEDLGVYF | |
| | GSSYVGYWGQGTTLTVSS | CSQSTHVPPWTFGGGTKLEIK | |
| | (SEQ ID No. 28) [Kabat] | (SEQ ID No. 29) [Kabat] | |
| 10C1-D9 | QVTLKESGPGILQSSQTLSLTCSFS | DIQMTQTTSSLSASLGDRVTISC | Domain 4 |
| | GFSLSTSDMGVSWIRQPSGKGLEWL | RASQDISNYLNWYQQKPDGTVKL | |
| | AHIYWDDDKRYNPSLKSRLTISKDA | LIYYTSRLHSGVPSRFSGSGSGT | |
| | SRNQVFLKIATVDTADTATYYCARS | DYSLTISNLEQEDIATYFCQQGN | |
| | PWIYYGHYWCFDVWGTGTTVTVSS | TLPFTFGSGTKLEIKR | |
| | (SEQ ID No. 30) [IMGT] | (SEQ ID No. 31) [IMGT] | |
| 15G7-2 | QVQLQQSGAELVKPGASVKLSCKAS | QIVLTQSPAIMSASPGEKVTMTC | Domain 4 |
| | GYTFTEYTIHWVKQRSGQGLEWIGW | SASSSVSYMYWYQQKPGSSPRLL | |
| | FYPGSGSIKYNEKFKDKATLTADKS | IYDTSNLASGVPVRFSGSGSGTS | |
| | SSTVYMELSRLTSEDSAVYFCARHG | YSLTISRMEAEDAATYYCQQWSS | |
| | DGYYLPPYYFDYWGQGTTLTVSS | YPLTFGAGTKLELK | |
| | (SEQ ID No. 32) [Kabat] | (SEQ ID No. 33) [Kabat] | |
| 2B12-8 | QVQLQQSGAELARPGASVKLSCKAS | DIVLTQSPATLSVTPGDSVSLSC | Domain 4 |
| | GYIFTSYGISWVKQRTGQGLEWIGE | RASQSISTNLHWYQQKSHASPRL | |
| | IYPRSGNTYYNEKFKGKATLTADKS | LIKYASQSVSGIPSRFSGSGSGT | |
| | SSTAYMELRSLTSEDSAVYFCARPI | DFTLSINSVETEDFGIFFCQQSY | |
| | YYGSREGFDYWGQGTTLTVSS | SWPYTFGGGTKLEIK | |
| | (SEQ ID No. 34) [Kabat] | (SEQ ID No. 35) [Kabat] | |
| 2C4-4 | QVQLQQPGAELVMPGASVKLSCKAS | DVLMTQTPLSLPVSLGDQASISC | Domain 5-7 |
| | GYTFTSYWMHWVKQRPGQGLEWIGE | RSSQSIVHSNGNTYLEWYLQKPG | |
| | IDPSDSYTNYNQKFKGKSTLTVDKS | QSPKLLIYKVSNRFSGVPDRFSG | |
| | SSTAYIQLSSLTSEDSAVYYCARWA | SESGTDFTLKISRVEAEDLGVYY | |
| | SYRGYAMDYWGQGTSVTVSS | CFQGSHVPWTFGGGTKLEIK | |
| | (SEQ ID No. 36) [Kabat] | (SEQ ID No. 37) [Kabat] | |
| 3E10-7 | EFQLQQSGPELVKPGASVKISCKAS | DIQMTQSPSSLSASLGERVSLTC | Domain 5-7 |
| | GYSFTDYNMNWVKQSNGKSLEWIGV | RASQEISGYLSWLQQKPDGTIKR | |
| | INPNYGTTSYNQRFKGKATLTVDQS | LIYAASTLDSGVPKRFSGSRSGS | |
| | SSTAYMQLNSLTSEDSAVYYCARSG | DYSLTISSLESEDFADYYCLQYA | |
| | LRYWYFDVWGTGTTVTVSS | SYPFTFGSGTKLEIK | |
| | (SEQ ID No. 38) [Kabat] | (SEQ ID No. 39) [Kabat] | |
| LT22 | EVQLVESGAEVKKPGSSVKVSCKAS | DIVMTQSPATLSVSPGERATLSC | Domain 5 |
| | GYTFTNYWINWVRQAPGQGLEWMGN | RSSQSLVHSNGNTYLHWYQQKPG | |
| | IYPSDSFTNYNQKFKDRVTITADKS | QAPRLLIYKVSNRFSGVPARFSG | |
| | TSTVYLELRNLRSDDTAVYYCTRDT | SGSGAEFTLTISSLQSEDFAVYY | |
| | QERSWYFDVWGQGTLVTVSS | CSQSTHVPWTFGQGTRLEIKR | |
| | (SEQ ID No. 40) {Kabat} | (SEQ ID No. 41) [Kabat] | |
| Inotuzumab | EVQLVQSGAEVKKPGASVKVSCKAS | DVQVTQSPSSLSASVGDRVTITC | Domain 7 |
| G5_44 | GYRFTNYWIHWVRQAPGQGLEWIGG | RSSQSLANSYGNTFLSWYLHKPG | |
| | INPGNNYATYRRKFQGRVTMTADTS | KAPQLLIYGISNRFSGVPDRFSG | |
| | TSTVYMELSSLRSEDTAVYYCTREG | SGSGTDFTLTISSLQPEDFATYY | |
| | YGNYGAWFAYWGQGTLVTVSS | CLQGTHQPYTFGQGTKVEIKR | |
| | (SEQ ID No. 42) [Kabat] | (SEQ ID No. 43) [Kabat] | |
| A7 (VHH) | QVQLQESGGGLVQAGGSLRLSCAAS | | Domains 1-3 |
| | GLTFSNYAMAWFRRAPGKERELVSR | | |
| | ISGRGTLTYYADSVKGRFTISRDND | | |
| | KNTVHLQMNSLKADDTAVYYCAAGS | | |
| | NSWGTRVVHTYDYWGQGTQVTVSS | | |
| | (SEQ ID No. 67) [IMGT] | | |
| B4 (VHH) | QVQLQQSGGGLVQAGGSLRLSCGAS | Domain 4 | |
| | GRTFSSLPMAWFRQAPGKEREFVAA | | |
| | ISGSGGATYYVDSVKGRFTISRDNA | | |
| | KNTVYLQMNSPKPEDTAVYYCAAKE | | |
| | GRFRWTYYTERFEYDSWGQGTQVTV | | |
| | SS | | |
| | (SEQ ID No. 68) [IMGT] | | |

An antigen binding domain of a chimeric receptor which binds to CD22 may comprise the CDRs from any of the CD22 antibodies listed in table 1. An antigen binding domain of a chimeric receptor which binds to CD22 may comprise the VH and/or VL sequence or VHH sequence from any of the CD22 antibodies listed in table 1, or a variant thereof which has at least 70, 80, 90 or 90% sequence identity, which variant retains the capacity to bind CD22.

BCMA

The B cell maturation target, also known as BCMA; TR17_HUMAN, TNFRSF17 (UniProt Q02223) is a transmembrane protein that is expressed in mature lymphocytes, e.g., memory B cells, plasmablasts and bone marrow plasma cells. BCMA is also expressed on myeloma cells. BCMA is a non-glycosylated type III transmembrane protein, which is involved in B cell maturation, growth and survival.

An antigen binding domain of a chimeric receptor which binds to BCMA may comprise a sequence derived from one of the commercially available anti-BCMA antibodies listed in the following table:

| Anti-BCMA | |
|---|---|
| antibody | Company |
| ab5972 | Abcam |
| ab54834 | Abcam |
| SG1 | Seattle Genetic Inc |
| LS-B2728 | LS Biosciences |
| LS-C18716 | LS Biosciences |
| LS-C357630 | LS Biosciences |
| LS-C53526 | LS Biosciences |
| LS-C196740 | LS Biosciences |

Alternatively it may comprise one of the following VH or VL sequences, or an scFv comprising a VH and VL sequence. The VH and VL sequences for three anti-BCMA antibodies are given below with CDR sequences underlined.

SEQ ID No. 44: antiBCMA Ab 1 VL
DIVLTQSPASLAMSLGKRATISCRASESVSVIGAHLIHWYQQKPGQPPKL
LIYLASNLETGVPARFSGSGSGTDFTLTIDPVEEDDVAIYSCLQSRIFPR
TFGGGTKLEIK
SEQ ID No. 45: antiBCMA Ab 1 VH
QIQLVQSGPELKKPGETVKISCKASGYTFTDYSINWVKRAPGKGLKWMGW
INTETREPAYAYDFRGRFAFSLETSASTAYLQINNLKYEDTATYFCALDY
SYAMDYWGQGTSVTVSS
SEQ ID No. 46: antiBCMA Ab 2 VH
QVQLVQSGAEVKKPGSSVKVSCKASGGTFSNYWMHWVRQAPGQGLEVVMG
ATYRGHSDTYYNQKFKGRVTITADKSTSTAYMELSSLRSEDTAVYYCARG
AIYDGYDVLDNWGQGTLVTVSS
SEQ ID No. 47: antiBCMA Ab 2 VL
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKR
SEQ ID No. 48: antiBCMA Ab 3 VH
EVQLVESGGGLVKPGRSLRLSCTASGFTFGDYALSWFRQAPGKGLEWVGV
SRSKAYGGTTDYAASVKGRFTISRDDSKSFAYLQMNSLKTEDTAVYYCCS
SGYSSGWTPFDYWGQGTLVTVSS
SEQ ID No. 49: antiBCMA Ab 3 VL
QSVLTQPPSASGTPGQRVTISCSGSSSNIGSNTVNWYQQLPGTAPKLLIF
NYHQRPSGVPDRFSGSKSGSSASLAISGLQSEDEADYYCAAWDDSLNGWV
FGGGTELTVLS
SEQ ID No. 50: antiBCMA Ab 4 VL
DVVMTQSHRFMSTSVGDRVSITCRASQDVNTAVSWYQQKPGQSPKLLIFS
ASYRYTGVPDRFTGSGSGADFTLTISSVQAEDLAVYYCQQHYSTPWTFGG
GTKLDIK
SEQ ID No. 51: antiBCMA Ab 4 VH
QIQLVQSGPDLKKPGETVKLSCKASGYTFTNFGMNWVKQAPGKGFKWMAW
INTYTGESYFADDFKGRFAFSVETSATTAYLQINNLKTEDTATYFCARGE
IYYGYDGGFAYWGQGTLVTVSA
SEQ ID No. 52: antiBCMA Ab 5 VL
DIVLTQSPPSLAMSLGKRATISCRASESVTILGSHLIYWYQQKPGQPPTL
LIQLASNVQTGVPARFSGSGSRTDFTLTIDPVEEDDVAVYYCLQSRTIPR
TFGGGTKLEIK
SEQ ID No. 53: antiBCMA Ab 5 VH
QIQLVQSGPELKKPGETVKISCKASGYTFRHYSMNWVKQAPGKGLKVVMG
RINTESGVPIYADDFKGRFAFSVETSASTAYLVINNLKDEDTASYFCSND
YLYSLDFWGQGTALTVSS

An antigen binding domain of a chimeric receptor which binds to BCMA may comprise the CDRs from antiBCMA Ab 1, 2 3, 4 or 5 described above.

An antigen binding domain of a chimeric receptor which binds to BCMA may comprise the VH and/or VL sequence from antiBCMA Ab 1, 2 3, 4 or 5 as described above, or a variant thereof which has at least 70, 80, 90 or 90% sequence identity, which variant retains the capacity to bind BCMA.

TACI

Transmembrane activator and calcium modulator and cyclophilin ligand (CAML) interactor) TACI (UniProtKB: O14836) is a regulator in immune responses, and like BCMA, is preferentially expressed in mature lymphocytes such as CD27+ memory B cells, especially marginal zone B cells, bone marrow plasma cells and myeloma cells.

An antigen binding domain or a chimeric receptor which binds to TACI may comprise a TACI binder derivable from one of the commercially available anti-TACI antibodies listed in the following table:

| Anti-TACI Ab | Company |
|---|---|
| 1A1 | BioLegend |
| ab5994 | Abcam |
| Ab79023 | Abcam |
| 11H3 | Affymetrix eBioscience |

Alternatively, it may comprise one of the following scFv sequences or a VH or VL domain derived therefrom.

anti-TACI scFv 1
SEQ ID No. 54
DIVMTQSQKFMSTTVGDRVSITCKASQNVGTAVAWYQQKPGQSPKWYSAS
NRYTGVPDRFTGSGSGTDFTLTISNMQSEDLADYFCQQYSSYRTFGGGTK
LEIKRSGGGGSGGGGSGGGGSQVTLKESGPGMLQPSQTLSLTCSFSGFSL
STFGMGVGWIRQPSGKGLEWLAHIVWVDDAQYSNPALRSRLTISKDTSKN
QVFLKIANVDTADTATYYCSRIHSYYSYDEGFAYWGQGTLVTVSS
anti-TACI scFv 2
SEQ ID No. 55
DIVMTQSQKFMSTTVGDRVTITCKASQNVGTAVAWYQQKPGQSPKWYSAS
NRYTGVPVRFTGSGSGTDFTLTINNMQSEDLADYFCQQYSSYPLTFGAGT
KLELKRSGGGGSGGGGSGGGGSQVQLKQSGPGLVAPSQSLSITCTVSGFS
LTSYGVDWVRQSPGKGLEWLGIIWGGGRTNYNSAFKSRLSISKDNSKSQV
FLKMNSLQTDDTAMYYCASGDRAADYWGQGTSVTVSS
anti-TACI scFv 3
SEQ ID No. 56
QAVVTQESALTTSPGETVTLTCRSSTGAVTTSDYAHWVQEKPDHLFTGLI
GGTNNRAPGVPARFSGSLIGDKAALTITGAQTEDEAIYFCALWYSNHWVF
GGGTKLTVLSGGGGSGGGGSGGGGSEVQLVESGGGLVKPGGSLKLSCAAS
GFTFSDYAMSWVRQTPEKRLEWVATISDGGTYTYYPDNIKGRFTISRDNT
KNNLYLQMSHLKSEDTAMYYCARYYGVYYAMGCWGQGTSVTVSS

Nucleic Acid

The present invention also provides a nucleic acid encoding a chimeric receptor of the invention.

For example, a nucleic acid encoding a tanCAR may have the structure:

AgB1-L-AgB2-spacer-TM-endo

in which
AgB1 is a nucleic acid sequence encoding a first antigen-binding domain;
L is a nucleic acid sequence encoding a linker;
AgB2 is a nucleic acid sequence encoding a second antigen-binding domain;
spacer is a nucleic acid sequence encoding a spacer;
TM is a nucleic acid sequence encoding a transmembrane domain; and
endo is a nucleic acid sequence encoding an intracellular signalling domain.

The antigen binding domain may, for example be an scFv or a domain antibody (dAb).

Nucleic Acid Construct

The present invention also provides a nucleic acid construct encoding a chimeric receptor of the invention.

Coiled-Coil Spacer Chimeric Receptor

A nucleic acid construct encoding a coiled-coil spacer CAR (FIG. 4D) may have the structure:

AgB1-CCS-TM1-endo1-coexpr-AgB2-CCS-TM2-endo2

in which
AgB1 is a nucleic acid sequence encoding the antigen-binding domain of the first polypeptide;
CCS is a nucleic acid sequence encoding a coiled-coil spacer;
TM1 is a nucleic acid sequence encoding the transmembrane domain of the first polypeptide; endol is a nucleic acid sequence encoding an intracellular signalling domain of the first polypeptide;
coexpr is a sequence allowing co-expression of the first and second polypeptides.

AgB2 is a nucleic acid sequence encoding the antigen-binding domain of the second polypeptide;

TM2 is a nucleic acid sequence encoding the transmembrane domain of the second polypeptide; endo2 is a nucleic acid sequence encoding an intracellular signalling domain of the second polypeptide.

Fab scFv/Fab dAb

A nucleic acid construct encoding a Fab scFv chimeric receptor (FIG. 4A) or a Fab dAb chimeric receptor (FIG. 5B) may have the structure:

AgB1-CH-TM-endo-coexpr-AgB2-CL

in which:
AgB1 is a nucleic acid sequence encoding the antigen-binding domain of the first polypeptide;
CH is a nucleic acid sequence encoding the heavy chain constant region of the first polypeptide;
TM is a nucleic acid sequence encoding a transmembrane domain of the first polypeptide;
endo is a nucleic acid sequence encoding an endodomain of the first polypeptide; coexpr is a nucleic acid sequence enabling co-expression of both first and second polypeptides;
AgB2 is a nucleic acid sequence encoding the antigen-binding domain of the second polypeptide; and
CL is a nucleic acid sequence encoding the light chain constant region of the second polypeptide.

A nucleic acid construct encoding a Fab scFv/dAb chimeric receptor may alternatively have the structure: AgB1-CL-TM-endo-coexpr-AgB2-CH

For both structures mentioned above, nucleic acid sequences encoding the two polypeptide may be in either order in the construct.

Dual Fab

A nucleic acid construct encoding a dual Fab chimeric receptor wherein each VH and VL are different (FIG. 4B) may have the structure:

VH1-CHi-S1-TM1-endo1-coexpr1-VL2-CL2-coexpr2-VH3-CHiii-53-TM3-endo3-coexpr3-VL4-CL4

in which:
VH1 is a nucleic acid sequence encoding the heavy chain variable region of the first polypeptide;
CHi is a nucleic acid sequence encoding the heavy chain constant region of the first polypeptide;
S1 is a nucleic acid sequence encoding a spacer of the first polypeptide;
TM1 is a nucleic acid sequence encoding a transmembrane domain of the first polypeptide;
endo1 is a nucleic acid sequence encoding an endodomain of the first polypeptide;
coexpr1, coexpr2 and coexpr3, which may be the same or different, are nucleic acid sequences enabling co-expression of adjacent polypeptides;
VL2 is a nucleic acid sequence encoding the light chain variable region of the second polypeptide;
CL2 is a nucleic acid sequence encoding the light chain constant region of the second polypeptide
VH3 is a nucleic acid sequence encoding the heavy chain variable region of the third polypeptide;
CHiii is a nucleic acid sequence encoding the heavy chain constant region of the third polypeptide;
S3 is a nucleic acid sequence encoding a spacer of the third polypeptide;
TM3 is a nucleic acid sequence encoding a transmembrane domain of the third polypeptide;
endo3 is a nucleic acid sequence encoding an endodomain of the third polypeptide;
VL4 is a nucleic acid sequence encoding the light chain variable region of the fourth polypeptide; and
CL4 is a nucleic acid sequence encoding the light chain constant region of the fourth polypeptide.

A nucleic acid construct encoding a Fab scFv chimeric receptor may alternatively have the structure: VL1-CL1-S1-TM1-endo1-coexpr1-VH2-CH2-coexpr2-VL3-CL3-53-TM3-endo3-coexpr3-VH4-CH4

For both structures mentioned above, nucleic acid sequences encoding the four polypeptides may be in any order in the construct.

A nucleic acid construct encoding a dual Fab chimeric receptor wherein the two VL domains are the same but the two VH domains are different (FIG. 5A) may have the structure:

VL1-CL1-S1-TM1-endo1-coexpr1-VH2-CH2-coexpr2-VH3-CH3

VL1 is a nucleic acid sequence encoding the light chain variable region of the second and fourth polypeptides;
CH1 is a nucleic acid sequence encoding the heavy chain constant region of the second and fourth polypeptides;
S1 is a nucleic acid sequence encoding a spacer of the second and fourth polypeptides;
TM1 is a nucleic acid sequence encoding a transmembrane domain of the second and fourth polypeptides;
endo1 is a nucleic acid sequence encoding an endodomain of the second and fourth polypeptides;
coexpr1, coexpr2 and coexpr3, which may be the same or different, are nucleic acid sequences enabling co-expression of adjacent polypeptides;
AgB2 is a nucleic acid sequence encoding the antigen-binding domain of the second polypeptide;
CL2 is a nucleic acid sequence encoding the light chain constant region of the second polypeptide
AgB3 is a nucleic acid sequence encoding the antigen-binding domain of the third polypeptide;
CH3 is a nucleic acid sequence encoding the heavy chain constant region of the third polypeptide;
S3 is a nucleic acid sequence encoding a spacer of the third polypeptide;
TM3 is a nucleic acid sequence encoding a transmembrane domain of the third polypeptide;
endo3 is a nucleic acid sequence encoding an endodomain of the third polypeptide; AgB4 is a nucleic acid sequence encoding the antigen-binding domain of the fourth polypeptide; and
CL4 is a nucleic acid sequence encoding the light chain constant region of the fourth polypeptide

In the above construct, the nucleic acid sequences encoding each polypeptide may be in any order in the construct.

Dual Fab scFv/Dual Fab dAb

A nucleic acid construct encoding a dual Fab scFv chimeric receptor (FIG. 4C) or a dual Fab dAb chimeric receptor (FIG. 5C) may have the structure:

AgB1-CH-S-TM-endo-coexpr-AgB2-CL, or

AgB1-CL-S-TM-endo-coexpr-AgB2-CH

in which:
AgB1 is a nucleic acid sequence encoding the first antigen binding domain;
CH is a nucleic acid sequence encoding the heavy chain constant region;
S is a nucleic acid sequence encoding a spacer;
TM is a nucleic acid sequence encoding a transmembrane domain;
Endo is a nucleic acid sequence encoding an endodomain;
Coexpr is a nucleic acid sequence enabling co-expression of the first and second polypeptides;
AgB2 is a nucleic acid sequence encoding the second antigen binding domain;
CL is a nucleic acid sequence encoding the light chain constant region;

For both structures mentioned above, nucleic acid sequences encoding the two polypeptides may be in either order in the construct.

Fc-Based Chimeric Receptors

A nucleic acid construct encoding a Fc scFv chimeric receptor (FIG. 7 A to C) or a Fc dAb chimeric receptor may have the structure:

AgB1-Fc1-TM1-endo1-coexpr-AgB2-Fc2-TM2-endo2

in which:
AgB1 is a nucleic acid sequence encoding the antigen-binding domain of the first polypeptide;
Fc1 is a nucleic acid sequence encoding the Fc domain of the first polypeptide;
TM1 is a nucleic acid sequence encoding a transmembrane domain of the first polypeptide;
Endo1 is a nucleic acid sequence encoding an endodomain of the first polypeptide;
coexpr is a nucleic acid sequence enabling co-expression of both first and second polypeptides;
AgB2 is a nucleic acid sequence encoding the antigen-binding domain of the second polypeptide; and
Fc2 is a nucleic acid sequence encoding the Fc domain of the second polypeptide;
TM2 is a nucleic acid sequence encoding a transmembrane domain of the second polypeptide;
Endo2 is a nucleic acid sequence encoding an endodomain of the second polypeptide

There may be a linker between the antigen binding domain and the Fc domain on the first and/or second polypeptide.

CH3-Based Chimeric Receptors

A nucleic acid construct encoding a CH3 scFv chimeric receptor (FIG. 8 A to C) or a Fc dAb chimeric receptor may have the structure:

AgB1-CH31-TM1-endo1-coexpr-AgB2-CH32-TM2-endo2

in which:
AgB1 is a nucleic acid sequence encoding the antigen-binding domain of the first polypeptide;
CH31 is a nucleic acid sequence encoding the CH3 domain of the first polypeptide;
TM1 is a nucleic acid sequence encoding a transmembrane domain of the first polypeptide;
Endo1 is a nucleic acid sequence encoding an endodomain of the first polypeptide; coexpr is a nucleic acid sequence enabling co-expression of both first and second polypeptides;
AgB2 is a nucleic acid sequence encoding the antigen-binding domain of the second polypeptide; and
CH32 is a nucleic acid sequence encoding the CH3 domain of the second polypeptide;
TM2 is a nucleic acid sequence encoding a transmembrane domain of the second polypeptide;
Endo2 is a nucleic acid sequence encoding an endodomain of the second polypeptide.

There may be a linker between the antigen binding domain and the CH3 domain on the first and/or second polypeptide.

Leucine Zipper Chimeric Receptors

A nucleic acid construct encoding a leucine zipper scFv chimeric receptor (FIG. 9A) or a leucine zipper dAb chimeric receptor may have the structure:

AgB1-Jun-TM1-endo1-coexpr-Ag B2-Fos-TM2-endo2

in which:
AgB1 is a nucleic acid sequence encoding the antigen-binding domain of the first polypeptide;
Jun is a nucleic acid sequence encoding a Jun leucine zipper domain of the first polypeptide;
TM1 is a nucleic acid sequence encoding a transmembrane domain of the first polypeptide;
Endo1 is a nucleic acid sequence encoding an endodomain of the first polypeptide; coexpr is a nucleic acid sequence enabling co-expression of both first and second polypeptides;
AgB2 is a nucleic acid sequence encoding the antigen-binding domain of the second polypeptide; and
Fos is a nucleic acid sequence encoding a Fos leucine zipper domain of the second polypeptide;
TM2 is a nucleic acid sequence encoding a transmembrane domain of the second polypeptide; Endo2 is a nucleic acid sequence encoding an endodomain of the second polypeptide.

There may be a linker between the antigen binding domain and the leucine zipper domain on the first and/or second polypeptide.

CD79a/b Chimeric Receptors

A nucleic acid construct encoding a CD79a/b scFv chimeric receptor (FIG. 9B) or a CD79a/b dAb chimeric receptor may have the structure:

AgB1-CD79a-TM1-endo1-coexpr-Ag B2-CD79b-TM2-endo2

in which:
AgB1 is a nucleic acid sequence encoding the antigen-binding domain of the first polypeptide;
CD79a is a nucleic acid sequence encoding a CD79a ectodomain;
TM1 is a nucleic acid sequence encoding a transmembrane domain of the first polypeptide;
Endo1 is a nucleic acid sequence encoding an endodomain of the first polypeptide;
coexpr is a nucleic acid sequence enabling co-expression of both first and second polypeptides;
AgB2 is a nucleic acid sequence encoding the antigen-binding domain of the second polypeptide; and
CD79b is a nucleic acid sequence encoding a CD79b ectodomain;
TM2 is a nucleic acid sequence encoding a transmembrane domain of the second polypeptide;
Endo2 is a nucleic acid sequence encoding an endodomain of the second polypeptide.

There may be a linker between the antigen binding domain and the CD79a/b domain on the first and/or second polypeptide.

Hybrid Chimeric Receptors

A nucleic acid construct encoding an scFv tanFab chimeric receptor (FIG. 6A) or a dAb tanFab chimeric receptor (FIG. 6B) may have the structure:

AgB1-VH-CH-TM-endo-coexpr-VL-CL

or

AgB1-VL-CL-TM-endo-coexpr-VH-CH

in which:
AgB1 is a nucleic acid sequence encoding the first antigen-binding domain;
VH is a nucleic acid sequence encoding a heavy chain variable domain of the second antigen binding domain;
CH is a nucleic acid sequence encoding a heavy chain constant region;
TM is a nucleic acid sequence encoding a transmembrane domain;
endo is a nucleic acid sequence encoding an endodomain;
coexpr is a nucleic acid sequence enabling co-expression of both first and second polypeptides;
VL is a nucleic acid sequence encoding a light chain variable domain of the second antigen binding domain; and
CL is a nucleic acid sequence encoding the light chain constant region.

There may be a linker between the first antigen binding domain and the VH/VL of the second antigen binding domain.

For both structures mentioned above, nucleic acid sequences encoding the two polypeptide may be in either order in the construct.

A nucleic acid construct encoding a dual variable tanFab chimeric receptor (FIG. 6C) may have the structure:

VH1-VH2-CH-TM-endo-coexpr-VL1-VL2-CL

or

VL1-VL2-CL-TM-endo-coexpr-VH1-VH2-CH

in which:
VH1 is a nucleic acid sequence encoding a heavy chain variable domain of the first antigen binding domain;
VH2 is a nucleic acid sequence encoding a heavy chain variable domain of the second antigen binding domain;
CH is a nucleic acid sequence encoding a heavy chain constant region;
TM is a nucleic acid sequence encoding a transmembrane domain;
endo is a nucleic acid sequence encoding an endodomain;
coexpr is a nucleic acid sequence enabling co-expression of both first and second polypeptides;
VL1 is a nucleic acid sequence encoding a light chain variable domain of the first antigen binding domain; and
VL2 is a nucleic acid sequence encoding a light chain variable domain of the second antigen binding domain
CL is a nucleic acid sequence encoding the light chain constant region.

The VH, VL and CH, CL domains may be mixed on the polypeptide, for example VL1-VH2-CL and VH1-VL2-CH. There may be a linker between the two VH/VL domains on a polypeptide.

For both structures mentioned above, nucleic acid sequences encoding the two polypeptide may be in either order in the construct.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No.57 (RAEGRGSLLTCGDVEENPGP).

Amino acid sequences for various constructs are shown below as SEQ ID No. 58 to 61.

Fc scFv Strand Exchange
aCD22_2C4-HCH2CH3pvaa_StrandExa-2A-aCD22_10C1-
HCH2CH3pvaa_StrandExb-CD28TM-41BBz
SEQ ID No. 58
METDTLLLWVLLLWVPGSTGDVLMTQTPLSLPVSLGDQASISCRSSQSIVH
SNGNTYLEWYLQKPGQSPKLLIYKVSNRFSGVPDRFSGSESGTDFTLKISR
VEAEDLGVYYCFQGSHVPWTFGGGTKLEIKRSGGGGSGGGGSGGGGSQVQL
QQPGAELVMPGASVKLSCKASGYTFTSYWMHWVKQRPGQGLEWIGEIDPSD
SYTNYNQKFKGKSTLTVDKSSSTAYIQLSSLTSEDSAVYYCARWASYRGYA
MDYWGQGTSVTVSSDPAEPKSPDKTHTCPPCPAPPVAGPSVFLFPPKPKDT
LMIARTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLP
PPSEELALNELVTLTCLVKGFYPSDIAVEWLQGSQELPREKYLTWAPVLDS
DGSFFLYSILRVAAEDWKKGDTFSCSVMHEALHNHYTQKSLDRSPGKKDPK
FWVLVVVGGVLACYSLLVTVAFIIFWVKRGRKKLLYIFKQPFMRPVQTTQE
EDGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRGK
GHDGLYQGLSTATKDTYDALHMQALPPRAEGRGSLLTCGDVEENPGPMETD
TLLLWVLLLWVPGSTGDIQMTQTTSSLSASLGDRVTISCRASQDISNYLNW
YQQKPDGTVKLLIYYTSRLHSGVPSRFSGSGSGTDYSLTISNLEQEDIATY
FCQQGNTLPFTFGSGTKLEIKRSGGGGSGGGGSGGGGSQVTLKESGPGILQ
SSQTLSLTCSFSGFSLSTSDMGVSWIRQPSGKGLEWLAHIYWDDDKRYNPS
LKSRLTISKDASRNQVFLKIATVDTADTATYYCARSPWIYYGHYWCFDVWG
TGTTVTVSSDPAEPKSPDKTHTCPPCPAPPVAGPSVFLFFPKPKDTLMISR
RPEVTCVVVDVSHEDPEVKFNWYVDGVEVHNAKTKPREEQYNSTYRVVSVL
TVLHQDWLNGKEYKCKVSNKALPAPIEKTISKAKGQPFRPEVHLLPPSREE
MTKNQVSLTCLARGFYPKDIAVEWESNGQPENNYKTTPSRQEPSQGTTTFA
VTSKLTVDKSRWQQGNVFSCSVMHEALHNHYTQKTISLSPGKKDPKFWVLV
VVGGCELRVKFSRSADAPAYQQGQNQLYNELNLGRREEYDVLDKRRGRDPE
MGGKPRRKNPQEGLYNELQKDKMAEAYSEIGMKGERRRGKGHDGLYQGLST
ATKDTYDALHMQALPPR*
Fc scFv Knob into holes
aCD22_2C4-HCH2CH3pvaa_KIHa-2A-aCD22_10C1-
HCH2CH3pvaa_KIHb-CD28TM-41BBz
SEQ ID No. 59
METDTLLLWVLLLWVPGSTGDVLMTQTPLSLPVSLGDQASISCRSSQSIVH
SNGNTYLEWYLQKPGQSPKLLIYKVSNRFSGVPDRFSGSESGTDFTLKISR
VEAEDLGVYYCFQGSHVPWTFGGGTKLEIKRSGGGGSGGGGSGGGGSQVQL
QQPGAELVMPGASVKLSCKASGYTFTSYWMHWVKQRPGQGLEWIGIEDPSD
SYTNYNQKFKGKSTLTVDKSSSTAYIQLSSLTSEDSAVYYCARWASYRGYA
MDYWGQGTSVTVSSDPAEPKSPDKTHTCPPCPAPPVAGPSVFLFPPKPKDT
LMIARTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVCTLP
PSRDELTKNQVSLSCAVKGFYPSDIAVEWESNGQPENNYKTTPPVLDSDGS
FFLVSKLTVDKSRWQQGNVFSCSVMHEALHNHYTQKSLSLSPGKKPDKFWV
LVVVGGVLACYSLLVTVAFIIFWVKRGRKKLLYIFKQPFMRPVQTTQEEDG
CSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHD
GLYQGLSTATKDTYDALHMQALPPRAEGRGSLLTCGDVEENPGPMETDTLL
LWVLLLWVPGSTGDIQMTQTTSSLSASLGDRVTISCRASQDISNYLNWYQQ
KPDGTVKLLIYYTSRLHSGVPSRFSGSGSGTDYSLTISNLEQEDIATYFCQ
QGNTLPFTFGSGTKLEIKRSGGGGSGGGGSGGGGSQVTLKESGPGILQSSQ
TLSLTCSFSGFSLSTSDMGVSWIRQPSGKGLEWLAHIYWDDDKRYNPSLKS
RLTISKDASRNQVFLKIATVDTADTATYYCARSPWIYYGHYWCFDVWGTGT
TVTVSSDPAEPKSPDKTHTCPPCPAPPVAGPSVFLFPPKPKDTLMIARTPE
VTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPCRDELTK
NQVSLWCLVKGFYPSDIAVEWESNGQPENNYKTTPPVLDSDGSFFLYSKLT
VDKSRWQQGNVFSCSVMHEALHNHYTQKSLSLSPGKKDPKFWVLVVVGGVL
ACYSLLVTVAFIIFWVKRGRKKLLYIFKQPFMRPVQTTQEEDGCSCRFPEE
EEGGCELRVKRSRSADAPAYQQGQNQLYNELNLGRREEYDVLDKRRGRDPE
MGGKPRRKNPQEGLYNELQKDKMAEAYSEIGMKGERRRGKGHDGLYQGLST
ATKDTYDALHMQALPPR*
Fc scFv Electrostatic steering
aCD22_2C4-HCH2CH3pvaa_ESa-2A-aCD22_10C1-
HCH2CH3pvaa_ESb-CD28TM-41BBz
SEQ ID No. 60
METDTLLLWVLLLWVPGSTGDVLMTQTPLSLPVSLGDQASISCRSSQSIVH
SNGNTYLEWYQKPGQSPKLLIYKVSNRFSGVPDRFSGSESGTDFTLKISRV
EAEDLGVYYCFQGSHVPWTFGGGTKLEIKRSGGGGSGGGGSGGGGSQVQLQ
QPGAELVMPGASVKLSCKASGYTFTSYWMHWVKQPRGQGLEWIGEIDPSDS
YTNYNQKFKGKSTLTVDKSSSTAYIQLSSLTSEDSAVYYCARWASYRGYAM
DYWGQGTSVTVSSDPAEPKSPDKTHTCPPCPAPPVAGPSVFLFPPKPKDTL
MIARTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPP
SRDELTKNQVSLTCLVKGFYPSDIAVEWESNGQPENNYDTTPPVLDSDGSF
FLYSDLTVDKSRWQQGNVFSCSVMHEALHNHYTQKSLSLSPGKKDPKFWVL
VVVGGVLACYSLLVTVAFIIFWVKRGRKKLLYIFKQPFMRPVQTTQEEDGC
SCRFPEEEEGGCELRVFKFSRSADAPAYQQGQNQLYNELNLGRREEYDVLD
KRRGRDPEMGGKPRRKNPQELGYNELQKDKMAEAYSEIGMKGERRRGKGHD
GLYQGLSTATKDTYDALHMQALPPRAEGRGSLLTCGDVEENPGPMMETDTL
LLWVLLLWVPGSTGDIQMTQTTSSLSASLGDRVTISVRASQDISNYLNWYQ
QKPDGTVKLLIYYTSRLHSGVPSRFSGSGSGTDYSLTISNLEQEDIATYFC
QQGNTLPFTFGSGTKLEIKRSGGGGSGGGGSGGGGSQVTLKESGPGILQSS
QTLSLTCSFSGFSLSTSDMGVSWIRQPSGKGLEWLAHIYWDDDKRYNPSLK
SRLTISKDASRNQVFLKIATVDTADTATYYCARSPWIYYGHYWCFDVWGTG
TTVTVSSDPAEPKSPDKTHTCPPCPAPPVAGPSVFLFPPKPKDTLMIARTP
EVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDKLT
KNQVSLTCLVKGFYPSDIAVEWESNGQPENNYKTTPPVLKSDGSFFLYSKL
TVDKSRWQQGNVFSCSVMHEALHNHYTQKSLSLSPGKKDPKFWVLVVVGGV
LACYSLLVTVAFIIFWVKRGRKKLLYIFKQPFMRPVQTTQEEDGCSCRFPE
EEEGGCELRVKFSRSADAPAYQQGQNQLYNELNLGRREEYDVLDKRRGRDP
EMGGKPRRKNPQEGLYNELQKDKMAEAYSEIGMKGERRRGKGDDGLYQGLS
TATKDTYDALHMQALPPR*
Dual variable Fab
aCD22_10C1VK-aCD22_2C4 VK-CK-2A-aCD22_10C1VH-
aCD22_2C4 VH-CH-Hinge-CD28TM-41BBz
SEQ ID No. 61
METDTLILWVLLLLVPGSTGDIQMTQTTSSLSASLGDRVTISCRASQDISN
YLNWYQQKPDGTVKLLIYYTSRLHSGVPSRFSGSGSGTDYSLTISNLEQED
IATYFCQQGNTLPFTFGSGTKLEIKRTVAAPSVFIFPPDVLMTQTPLSLPV
SLGDQASISVRSSQSIVHSNGNTYLEWYLQKPGQSPKLLIYKVSNRFSGVP
DRFSGSESGTDFTLKISRVEAEDLGVYYCFQGSHVPWTFGGGTKLEIKRTV
AAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRG
ECRAATNFSLLKQAGDVEENPGPMGWSCIILFLVATATGVHSQVTLKESGP
GILQSSQTLSLTCSFSGFSLSTSDMGVSWIRQPSGKGLEWLAHIYWDDDKR
YNPSLKSRLTISKDASRNQVFLKIATVDTADTATYYCARSPWIYYGHYWCF
DVWGTGTTVTVSSASTKGPSVFPLAPQVQLQQPGAELVMPGASVKLSCKAS
GYTFTSYWMHWVKQRPGQGLEWIGEIDPSDSYTNYNQKFKGKSTLTVDKSS
STAYIQLSSLTSEDSAVYYCARWASYRGYAMDYWGQGTSVTVSSASTKGPS
VPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCP
PCPKDPKFWVLVVVGGVLACYSLLVTVAFIIFWVKRGRKKLLYIFKQPFMR
PVQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGMK
GERRRGKGHDGLYQGLSTATKDTYDALHMQALPPR

Vector

The present invention also provides a vector, or kit of vectors, which comprises one or more nucleic acid sequence(s) encoding a chimeric receptor according to the invention. Such a vector may be used to introduce the nucleic acid sequence(s) into a host cell so that it expresses a chimeric polypeptide according to the first aspect of the invention.

The vector may, for example, be a plasmid or a viral vector, such as a retroviral vector or a lentiviral vector, or a transposon based vector or synthetic mRNA.

The vector may be capable of transfecting or transducing a T cell or a NK cell.

Cell

The present invention provides a cell which comprises a chimeric receptor of the invention.

The cell may comprise a nucleic acid or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+ FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

T or NK cells according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the chimeric polypeptide according to the first aspect of the invention, for example by treatment with an anti-CD3 monoclonal antibody.

The T or NK cell of the invention may be made by:
(i) isolation of a T or NK cell-containing sample from a subject or other sources listed above; and
(ii) transduction or transfection of the T or NK cells with one or more a nucleic acid sequence(s) encoding a chimeric polypeptide.

The T or NK cells may then by purified, for example, selected on the basis of expression of the antigen-binding domain of the antigen-binding polypeptide.

Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a T or NK cell-containing sample;
(ii) transducing or transfecting such cells with a nucleic acid sequence or vector provided by the present invention;
(iii) administering the cells from (ii) to a subject.

The T or NK cell-containing sample may be isolated from a subject or from other sources, for example as described above. The T or NK cells may be isolated from a subject's own peripheral blood (1st party), or in the setting of a haematopoietic stem cell transplant from donor peripheral blood (2nd party), or peripheral blood from an unconnected donor (3rd party).

The present invention provides a chimeric polypeptide-expressing cell of the present invention for use in treating and/or preventing a disease.

The invention also relates to the use of a chimeric polypeptide-expressing cell of the present invention in the manufacture of a medicament for the treatment and/or prevention of a disease.

The disease to be treated and/or prevented by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma or T-cell acute Lymphoblastic Leukaema (T-ALL).

The cells of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

The cells and pharmaceutical compositions of present invention may be for use in the treatment and/or prevention of the diseases described above.

The invention will now be further described by way of Examples, which are meant to serve to assist one of ordinary skill in the art in carrying out the invention and are not intended in any way to limit the scope of the invention.

### EXAMPLES

### Example 1: Bivalent Fab CARs Targeting CD22

T cells were either left untransduced or transduced with a vector encoding one of the chimeric receptors listed below.

The chimeric receptors are “Fab scFvs”, which are made up of a first chain comprising a scFv targeting a first epitope of CD22 with a CH1 spacer domain, Tyrp transmembrane domain and 41BB and CD3 zeta signalling domains; and a second chain comprising a scFv against a second epitope of CD22 followed by a CL domain without a transmembrane domain. The two chains form a heterodimer with specificities to two separate epitopes. The binder 2C4 targets a membrane distal epitope of CD22, whereas the binder 3B4 targets a membrane proximal epitope.

Vector 1: SFGmR.RQR8-2A-aCD22_2C4_LH-CH-TyrpTM-41BBz-2A-aCD22_2B12_LH-CL

Vector 2: SFGmR.RQR8-2A-aCD22_Inotuzmab_LH-CH-TyrpTM-41BBz-2A-aCD22_2B12_LH-CL

Vector 3: SFGmR.RQR8-2A-aCD22_LT22_LH-CH-2A-TyrpTM-41BBz-aCD22_2B12_LH-CL

Vector 4: SFGmR.RQR8-2A-aCD22_2C4_LH-CH-2A-TyrpTM-41BBz-aCD22_10C1_LH-CL

Vector 5: SFGmR.RQR8-2A-aCD22_Inotuzmab_LH-CH-TyrpTM-41BBz-2A-aCD22_2B12_LH-CL

Vector 6: SFGmR.RQR8-2A-aCD22_LT22_LH-CH-2A-TyrpTM-41BBz-aCD22_2B12_LH-CL

Vector 7: SFGmR.RQR8-2A-aCD22_2C4_LH-CH-2A-TyrpTM-41BBz-aCD22_7 G6_LH-CL

Vector 8: SFGmR.RQR8-2A-aCD22_Inotuzmab_LH-CH-TyrpTM-41BBz-2A-aCD22_7 G6_LH-CL

Vector 9: SFGmR.RQR8-2A-aCD22_LT22_LH-CH-2A-TyrpTM-41BBz-aCD22_7 G6_LH-CL

Seven days after the thawing of PBMCs, the culture is depleted of CD56 NK cells to reduce background cytotoxicity. On the eighth day, the T-cells are co-cultured with Raji target cells at a ratio 1:1.

The assay is carried out in a 96-well plate in 0.2 ml total volume using 5×10⁴ transduced T-cells per well and an equal number of target cells. The co-cultures are set up after being normalised for the transduction efficiency. A FACS-based killing assay is carried out after 72 h of incubation.

Secretion of cytokines such as IFN-γ and IL-2 after 72 hrs incubation is also investigated using a cytokine bead array.

### Example 2: Bivalent FabCAR Targeting TACI

T cells were either left untransduced or transduced with a vector encoding a “Fab scFv” chimeric receptor against TACI. The chimeric receptor is made up of a first chain comprising a scFv targeting a first epitope of TACI with a CH1 spacer domain, Tyrp transmembrane domain and 41BB and CD3 zeta signalling domains; and a second chain comprising a scFv against a second epitope of TACI followed by a CL domain without a transmembrane domain. The two chains form a heterodimer with specificities to two separate epitopes. The binders 2H6 and 2G2 target distinct epitopes on TACI.

Vector 10: SFGmR.RQR8-2A-aTACI_2G2_LH-CH-2A-TyrpTM-41 BBz-aTACI_2H6_LH-CL

Seven days after the thawing of PBMCs, the culture is depleted of CD56 NK cells to reduce background cytotoxicity. On the eighth day, the T-cells are co-cultured with target cells at a ratio 1:1. T cells are co-cultured with a panel of target cells, as follows:

1. Non-transduced SupT1 cells (control)

2. TACI Low-SupT1 (expressing approximately 500 copies TACI per cell)

3. TACI High-SupT1 (expressing 1000-2000 copies TACI per cell)

4. MM1.S—a multiple myeloma cell line used as a positive control

The assay is carried out in a 96-well plate in 0.2 ml total volume using 5×10⁴ transduced T-cells per well and an equal number of target cells. The co-cultures are set up after being normalised for the transduction efficiency. A FACS-based killing assay is carried out after 72 h of incubation.

Secretion of cytokines such as IFN-γ and IL-2 after 72 hrs incubation is also investigated using a cytokine bead array.

### Example 3: Bivalent FabCAR Targeting BCMA

T cells were either left untransduced or transduced with a vector encoding a “Fab scFv” chimeric receptor against BCMA. The chimeric receptor is made up of a first chain comprising a scFv targeting a first epitope of BCMA with a CH1 spacer domain, Tyrp transmembrane domain and 41BB and CD3 zeta signalling domains; and a second chain comprising a scFv against a second epitope of BCMA followed by a CL domain without a transmembrane domain. The two chains form a heterodimer with specificities to two separate epitopes.

Vector 11: SFGmR.RQR8-2A-aBCMA1_LH-CH-2A-TyrpTM-41BBz-aBCMA4_LH-CL

Seven days after the thawing of PBMCs, the culture is depleted of CD56 NK cells to reduce background cytotoxicity. On the eighth day, the T-cells are co-cultured with target cells at a ratio 1:1. T cells are co-cultured with a panel of target cells, as follows:

1. Non-transduced SupT1 cells (control)

2. BCMA Low-SupT1 (expressing approximately 500 copies BCMA per cell)

3. BCMA High-SupT1 (expressing 1000-2000 copies BCMA per cell)

4. MM1.S—a multiple myeloma cell line used as a positive control

The assay is carried out in a 96-well plate in 0.2 ml total volume using 5×10⁴ transduced T-cells per well and an equal number of target cells. The co-cultures are set up after being normalised for the transduction efficiency. A FACS-based killing assay is carried out after 72 h of incubation.

Secretion of cytokines such as IFN-γ and IL-2 after 72 hrs incubation is also investigated using a cytokine bead array.

### Example 4: Bivalent FabCAR Targeting TACI

T cells were either left untransduced or transduced with a vector encoding a “Fab scFv” chimeric receptor against TACI. The chimeric receptor is made up of a first chain comprising a scFv targeting a first epitope of TACI with a CH1 spacer domain, Tyrp transmembrane domain and 41BB and CD3 zeta signalling domains; and a second chain comprising a scFv against a second epitope of TACI followed by a CL domain without a transmembrane domain. The two chains form a heterodimer with specificities to two separate epitopes.

Vector 12: SFGmR.RQR8-2A-aTACI1_LH-CH-2A-TyrpTM-41BBz-aTACI2_LH-CL

Seven days after the thawing of PBMCs, the culture is depleted of CD56 NK cells to reduce background cytotoxicity. On the eighth day, the T-cells are co-cultured with target cells at a ratio 1:1. T cells are co-cultured with a panel of target cells, as follows:

1. Non-transduced SupT1 cells (control)

2. TACI Low-SupT1 (expressing approximately 500 copies TACI per cell)

3. TACI High-SupT1 (expressing 1000-2000 copies TACI per cell)

4. MM1.S—a multiple myeloma cell line used as a positive control

The assay is carried out in a 96-well plate in 0.2 ml total volume using 5×10⁴ transduced T-cells per well and an equal number of target cells. The co-cultures are set up after being normalised for the transduction efficiency. A FACS-based killing assay is carried out after 72 h of incubation.

Secretion of cytokines such as IFN-γ and IL-2 after 72 hrs incubation is also investigated using a cytokine bead array.

### Example 5: Design, Construction and Cytotoxicity of conCAT CARs Targeting CD22

Two different conCAT CARs were constructed targeting CD22, based on a COMP spacer format (FIG. 4D).

The first conCAT CAR comprise a first antigen-binding domain derived from 1D9 (i.e. 1D9-3 as shown in Table 1 with a VH sequence shown as SEQ ID No. 18 and a VL sequence shown as SEQ ID No. 19) and 10C1 (i.e. 10C1-D9 as shown in Table 1 with a VH sequence shown as SEQ ID No. 30 and a VL sequence shown as SEQ ID No. 31).

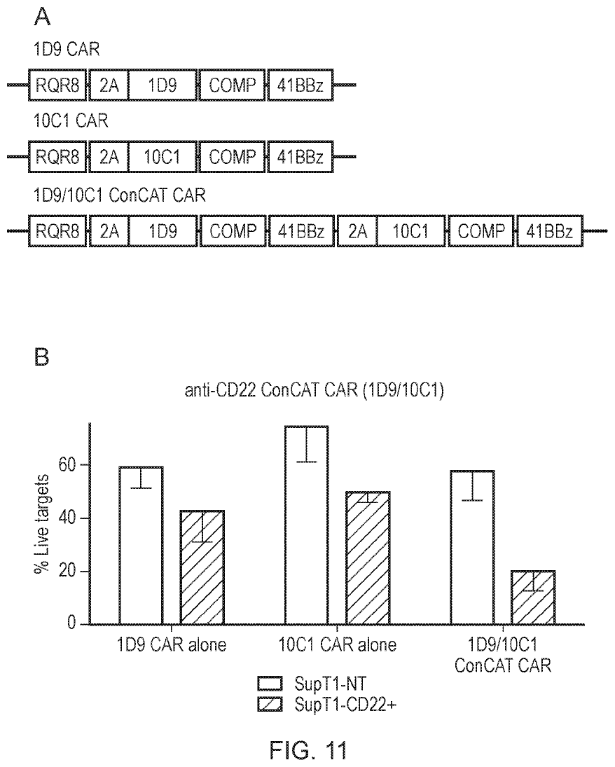

The constructs used to produce 1D9 (single) CAR, 10C1 CAR and 1D9/10C1 ConCAT CAR as shown in FIG. 11A.

The second conCAT CAR comprise a first antigen-binding domain derived from g5_44 (i.e. Inotuzumab as shown in Table 1 with a VH sequence shown as SEQ ID No. 42 and a VL sequence shown as SEQ ID No. 43) and 10C1 (i.e. 10C1-D9 as shown in Table 1).

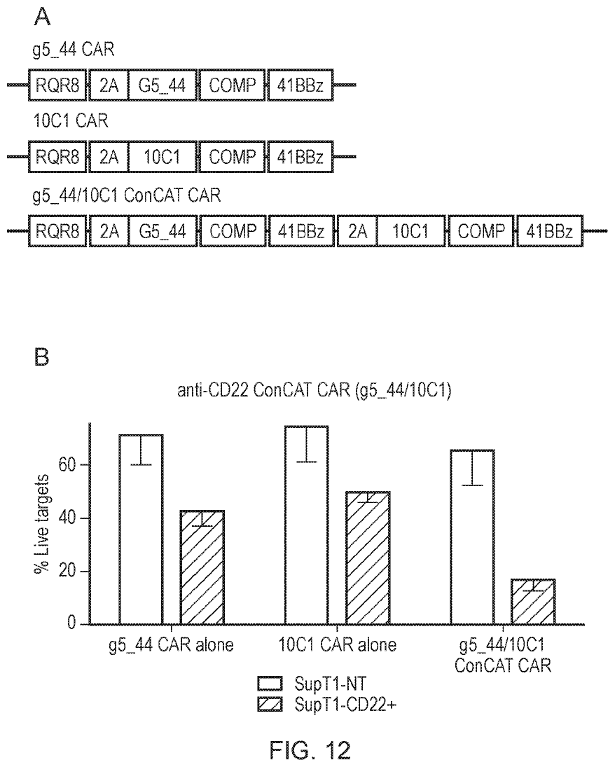

The constructs used to produce g5_44 (single) CAR, 10C1 CAR and g5_44/1001 ConCAT CAR as shown in FIG. 12A.

It was then investigated whether ConCAT CARs having two different antigen-binding domains which bind to different epitopes of CD22 are more efficacious at killing CD22 positive target cells that CAR-T cells expressing single binders alone. Lymphocytes were transduced with vectors comprising the various constructs shown in FIG. 11A and FIG. 12A. Three days post transduction, expression of marker (RQR8) and ligand (CD22) binding profiles of each CAR were determined by staining with anti-RQR8-APC and hCD22-FITC, respectively to determine the transduction efficiency. Following transduction, all samples were CD56 depleted using negative bead selection. Transduced cells were co-cultured with SupT1-NT and SupT1-CD22+ target cells at an effector to target ratio of 1:1. Cytotoxicity readout was taken 24 hours post co-culture by staining with anti-CD3-PeCy7 to differentiate effector T-cells and target cells. Data was obtained from two donors are cytotoxicity was normalized to non-transduced T-cells. The results are shown in FIG. 11B and FIG. 12B.

For both the 1D9/10C1 ConCAT CAR (FIG. 11B) and the g5_44/1001 ConCAT CAR (FIG. 12B) superior killing of CD22 positive target cells was seen with the conCAT CAR than with a CAR expressing either single binder.

Methology

Cell Lines

SupT1 cell line (NT and CD22+) were cultured in RPMI-1640 medium supplemented with 10% fetal bovine serum (FBS) and 1% GlutaMAX. T-cells were isolated from peripheral blood mononuclear cells (PBMCs) and maintained in RPMI-1640 Medium supplemented with 10% FBS, 1% GlutaMAX and 100 U/mL IL-2.

Transduction

Retrovirus was generated by transiently transfecting HEK293T cells using GeneJuice with RDF plasmid (RD114 envelope), gag/pol plasmid and CAR plasmid. Retroviral viral supernatant was harvested at 48 and 72 hours. T cells were stimulated using 0.5 μg/mL of anti-CD3 and anti-CD28 antibodies in T175 TC-treated flasks and maintained in 100 U/mL IL-2. Non-TC treated six-well plates were coated with Retronectin in accordance to manufacturers instructions (Takara Bio) and incubated at 4° C. for 24 hours prior to T cell transduction. 3 ml of viral supernatant was plated prior to the addition of 1 ml of activated T cells at a concentration of 1×10 cells/ml, 100 U/mL of IL-2 was then added and centrifuged at 1000×g for 40 minutes at room temperature and incubated at 37° C. and 5% CO₂ for 2-3 days.

NK Cells and NKT Cells Depletion

EasySepTM Human CD56 Positive Selection Kit was used to carry out CD56 depletion.

Cytotoxicity Assay

CAR T-cells were co-cultured with SupT1-NT and SupT1-CD22 at effector to target ratio of 1:1 (50,000:50,000 cells) in a TC-treated 96-well plate. Readout was taken 24 hours post co-culture by staining with anti-CD3-PeCy7 to differentiate effector T-cells and target cells, SYTOX Blue dead cell stain (S34857) was used to exclude dead cells. Cytotoxicity readouts were acquired using the MACSQuant® Analyzer 10 flow cytometer.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A chimeric receptor which binds a target antigen on a target cell, which comprises:
a first antigen binding domain which binds a first epitope of the target antigen, a second antigen binding domain which binds a second epitope of the target antigen;
a transmembrane domain; and
an intracellular signalling domain
wherein the chimeric receptor does not simultaneously bind the first epitope and the second epitope of the same target antigen molecule.

2. A chimeric receptor according to claim 1, which comprises first and second polypeptides, wherein:
the first polypeptide comprises the first antigen binding domain;
the second polypeptide comprises the second antigen binding domain;
the first and/or second polypeptide comprises a transmembrane domain; and
the first and second polypeptides associate to form the chimeric receptor.

3. A chimeric receptor according to claim 2, wherein
the first polypeptide comprises a heavy chain constant region; and
the second polypeptide comprises a light chain constant region.

4. (canceled)

5. A chimeric receptor according to claim 2 wherein the first and second polypeptides have the general structure:
ABD-CC-TM
in which ABD is the antigen binding domain, CCS is a coiled-coil spacer domain and TM is a transmembrane domain.

6. A chimeric receptor according to claim 2, wherein the first and second polypeptides comprise an engineered CH3 domain.

7. (canceled)

8. A chimeric receptor according to claim 1, which comprises two polypeptides, wherein one polypeptide comprises a heavy chain variable region (VH) and the other comprises a light chain variable region (VL) which associate to form the first antigen binding domain.

9. (canceled)

10. A chimeric receptor according to claim 8, which comprises four polypeptides:
(i) a first polypeptide which comprises a first heavy chain variable region (VH) and a first heavy chain constant region;
(ii) a second polypeptide which comprises a first light chain variable region (VL) and a first light chain constant region;
(iii) a third polypeptide which comprises a second heavy chain variable region (VH) and a second heavy chain constant region; and
(iv) a fourth polypeptide which comprises a second light chain variable region (VL) and a second light chain constant region;
wherein
the first VH and first VL associate to form the first antigen binding domain;
the second VH and second VL associate to form the second antigen binding domain;
the first and/or second polypeptide chain comprise(s) a transmembrane domain; and
the third and/or fourth polypeptide chain comprise(s) a transmembrane domain.

11. A chimeric receptor according to claim 10, wherein the first VL and the second VL are the same, but the first VH is different from the second VH.

12. A chimeric receptor according to claim 1 in which the first and second antigen binding domains are linked on a single polypeptide chain.

13. (canceled)

14. A chimeric receptor according to claim 1, wherein the first epitope is a membrane proximal epitope and the second epitope is a membrane distal epitope, or vice versa.

15. A chimeric receptor according to claim 1, wherein the target antigen is B cell maturation antigen (BCMA), transmembrane activator and calcium modulator and cyclophilin ligand interactor (TACI), CD22 or CD21.

16. A cell which comprises a chimeric receptor according to claim 1.

17. A nucleic acid sequence encoding a chimeric receptor according to claim 1.

18-20. (canceled)

21. A vector comprising a nucleic acid sequence according to claim 17.

22-25. (canceled)

26. A method for making a cell according to claim 16, which comprises the step of introducing: a nucleic acid sequence encoding a chimeric receptor into a cell.

27. (canceled)

28. A pharmaceutical composition comprising a plurality of cells according to claim 16.

29. A method for treating and/or preventing a disease, which comprises the step of administering a pharmaceutical composition according to claim 28 to a subject.

30. A method according to claim 29, which comprises the following steps:
(i) isolation of a cell-containing sample from a subject;
(ii) transduction or transfection of the cells with: a nucleic acid encoding a chimeric receptor; and
(iii) administering the cells from (ii) to the subject.

31. A method according to claim 29, wherein the disease is a cancer.

32-33. (canceled)


SEQUENCE LISTING (72 sequences)

SEQ ID NO: 1 - protein, 106 aa, Artificial Sequence
  kappa chain constant domain
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 97 aa, Artificial Sequence
  CH domain from a gamma immunoglobulin heavy chain
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 3 - protein, 20 aa, Artificial Sequence
  spacer, human IgG1 hinge
AEPKSPDKTHTCPPCPKDPK

SEQ ID NO: 4 - protein, 217 aa, Artificial Sequence
  Heavy chain CH2CH3
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 107 aa, Artificial Sequence
  Heavy chain CH3
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 216 aa, Artificial Sequence
  knob-into-hole Fc sequence, HCH2CH3pvaa_KIHa
APPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 7 - protein, 216 aa, Artificial Sequence
  knob-into-hole Fc sequence, HCH2CH3pvaa_KIHb
APPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 219 aa, Artificial Sequence
  strand exchange Fc sequence, HCH2CH3pvaa_StrandExa
APPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGK

SEQ ID NO: 9 - protein, 219 aa, Artificial Sequence
  strand exchange Fc sequence, HCH2CH3pvaa_StrandExb
APPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 10 - protein, 216 aa, Artificial Sequence
  charge pair Fc sequence, HCH2CH3pvaa_ESa
APPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 11 - protein, 216 aa, Artificial Sequence
  charge pair Fc sequence, HCH2CH3pvaa_ESb
APPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 12 - protein, 111 aa, Artificial Sequence
  spacer, CD79a ectodomain
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNR

SEQ ID NO: 13 - protein, 131 aa, Artificial Sequence
  spacer, CD79b ectodomain
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKD

SEQ ID NO: 14 - protein, 39 aa, Artificial Sequence
  spacer, Fos-Leucine zipper
LTATLQAETDQLEDEKSALQTEIANLLKEKEKLEFILAA

SEQ ID NO: 15 - protein, 35 aa, Artificial Sequence
  spacer, Jun-Leucine zipper
LEEKVKTLKAQNSELASTANMLREQVAQLKQKVMN

SEQ ID NO: 16 - protein, 45 aa, Artificial Sequence
  cartilage-oligomeric matrix protein (COMP) coiled coil domain
DLGPQMLRELQETNAALQDVRELLRQQVREITFLKNTVMECDACG

SEQ ID NO: 17 - protein, 15 aa, Artificial Sequence
  hinge spacer
EPKSCDKTHTCPPCP

SEQ ID NO: 18 - protein, 119 aa, Artificial Sequence
  antibody 1D9-3, VH sequence
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVVDYLYAMDYWGQGTSVTVSS

SEQ ID NO: 19 - protein, 108 aa, Artificial Sequence
  antibody 1D9-3, VL sequence
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPFTFGSGTKLEIKR

SEQ ID NO: 20 - protein, 120 aa, Artificial Sequence
  antibody 3B4-13, VH sequence
QVQLQQSGAELVRPGASVTLSCKASGYTFTDYEMHWVKQTPVHGLEWIGAIDPETGATAYNQKFKGKAILTADKSSSTAYMDLRSLTSEDSAVYYCTRYDYGSSPWFAYWGQGTLVTVSA

SEQ ID NO: 21 - protein, 109 aa, Artificial Sequence
  antibody 3B4-13, VL sequence
QAVVTQESALTTSPGETVTLTCRSSAGAVTTSNYANWVQEKPDHLFTGLIGGTNNRAPGVPARFSGSLIGDKAALTITGAQTEDEAIYFCALWNSNHWVFGGGTKLTVL

SEQ ID NO: 22 - protein, 119 aa, Artificial Sequence
  antibody 7G6-6, VH sequence
QVQLQQPGAELVMPGASVKLSCKASGYTFTSYWMHWVKQRPGQGLEWIGEIDPSDSYTNYNQKFKGKATLTVDKSSSTAYMQLSSLTSEDSAVYYCARGYYGSSSFDYWGQGTTLTVSS

SEQ ID NO: 23 - protein, 111 aa, Artificial Sequence
  antibody 7G6-6, VL sequence
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYTFGGGTKLEIK

SEQ ID NO: 24 - protein, 120 aa, Artificial Sequence
  antibody 6C4-6, VH sequence
QVQLKESGPGLVAPSQSLSITCTVSGFSLTSYGVHWVRQPPGKGLEWLVVIWSDGSTTYNSALKSRLSISKDNSKSQVFLKMNSLQTDDTAMYYCARHADDYGFAWFAYWGQGTLVTVSA

SEQ ID NO: 25 - protein, 107 aa, Artificial Sequence
  antibody 6C4-6, VL sequence
DIQMTQSPASLSASVGETVTITCRASENIYSYLAWYQQKQGKSPQLLVYNAKTLAEGVPSRFSGSGSGTQFSLKINSLQPEDFGSYYCQHHYGTPPTFGGGTKLEIK

SEQ ID NO: 26 - protein, 121 aa, Artificial Sequence
  antibody 4D9-12, VH sequence
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARSSTTVVDWYFDVWGTGTTVTVSS

SEQ ID NO: 27 - protein, 107 aa, Artificial Sequence
  antibody 4D9-12, VL sequence
DIQMTQSPSSLSASLGERVSLTCRASQEISGYLSWLQQKPDGTIKRLIYAASTLDSGVPKRFSGSRSGSDYSLTISSLESEDFADYYCLQYASYPFTFGSGTKLEIK

SEQ ID NO: 28 - protein, 118 aa, Artificial Sequence
  antibody 5H4-9, VH sequence
QVQVQQPGAELVRPGTSVKLSCKASGYTFTRYWMYWVKQRPGQGLEWIGVIDPSDNFTYYNQKFKGKATLTVDTSSSTAYMQLSSLTSEDSAVYYCARGYGSSYVGYWGQGTTLTVSS

SEQ ID NO: 29 - protein, 113 aa, Artificial Sequence
  antibody 5H4-9, VL sequence
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIK

SEQ ID NO: 30 - protein, 124 aa, Artificial Sequence
  antibody 10C1-D9, VH sequence
QVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSS

SEQ ID NO: 31 - protein, 108 aa, Artificial Sequence
  antibody 10C1-D9, VL sequence
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIKR

SEQ ID NO: 32 - protein, 123 aa, Artificial Sequence
  antibody 15G7-2, VH sequence
QVQLQQSGAELVKPGASVKLSCKASGYTFTEYTIHWVKQRSGQGLEWIGWFYPGSGSIKYNEKFKDKATLTADKSSSTVYMELSRLTSEDSAVYFCARHGDGYYLPPYYFDYWGQGTTLTVSS

SEQ ID NO: 33 - protein, 106 aa, Artificial Sequence
  antibody 15G7-2, VL sequence
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELK

SEQ ID NO: 34 - protein, 121 aa, Artificial Sequence
  antibody 2B12-8, VH sequence
QVQLQQSGAELARPGASVKLSCKASGYIFTSYGISWVKQRTGQGLEWIGEIYPRSGNTYYNEKFKGKATLTADKSSSTAYMELRSLTSEDSAVYFCARPIYYGSREGFDYWGQGTTLTVSS

SEQ ID NO: 35 - protein, 107 aa, Artificial Sequence
  antibody 2B12-8, VL sequence
DIVLTQSPATLSVTPGDSVSLSCRASQSISTNLHWYQQKSHASPRLLIKYASQSVSGIPSRFSGSGSGTDFTLSINSVETEDFGIFFCQQSYSWPYTFGGGTKLEIK

SEQ ID NO: 36 - protein, 120 aa, Artificial Sequence
  antibody 2C4-4, VH sequence
QVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSS

SEQ ID NO: 37 - protein, 112 aa, Artificial Sequence
  antibody 2C4-4, VL sequence
DVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIK

SEQ ID NO: 38 - protein, 119 aa, Artificial Sequence
  antibody 3E10-7, VH sequence
EFQLQQSGPELVKPGASVKISCKASGYSFTDYNMNWVKQSNGKSLEWIGVINPNYGTTSYNQRFKGKATLTVDQSSSTAYMQLNSLTSEDSAVYYCARSGLRYWYFDVWGTGTTVTVSS

SEQ ID NO: 39 - protein, 107 aa, Artificial Sequence; identical to SEQ ID NO: 27
  antibody 3E10-7, VL sequence

SEQ ID NO: 40 - protein, 120 aa, Artificial Sequence
  antibody LT22, VH sequence
EVQLVESGAEVKKPGSSVKVSCKASGYTFTNYWINWVRQAPGQGLEWMGNIYPSDSFTNYNQKFKDRVTITADKSTSTVYLELRNLRSDDTAVYYCTRDTQERSWYFDVWGQGTLVTVSS

SEQ ID NO: 41 - protein, 113 aa, Artificial Sequence
  antibody LT22, VL sequence
DIVMTQSPATLSVSPGERATLSCRSSQSLVHSNGNTYLHWYQQKPGQAPRLLIYKVSNRFSGVPARFSGSGSGAEFTLTISSLQSEDFAVYYCSQSTHVPWTFGQGTRLEIKR

SEQ ID NO: 42 - protein, 121 aa, Artificial Sequence
  antibody Inotuzumab G5_44, VH sequence
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 43 - protein, 113 aa, Artificial Sequence
  antibody Inotuzumab G5_44, VL sequence
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 44 - protein, 111 aa, Artificial Sequence
  antiBCMA Ab 1 VL
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 45 - protein, 117 aa, Artificial Sequence
  antiBCMA Ab 1 VH
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 46 - protein, 121 aa, Artificial Sequence
  antiBCMA Ab 2 VH
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 47 - protein, 108 aa, Artificial Sequence
  antiBCMA Ab 2 VL
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 48 - protein, 123 aa, Artificial Sequence
  antiBCMA Ab 3 VH
EVQLVESGGGLVKPGRSLRLSCTASGFTFGDYALSWFRQAPGKGLEWVGVSRSKAYGGTTDYAASVKGRFTISRDDSKSFAYLQMNSLKTEDTAVYYCCSSGYSSGWTPFDYWGQGTLVTVSS

SEQ ID NO: 49 - protein, 111 aa, Artificial Sequence
  antiBCMA Ab 3 VL
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTELTVLS

SEQ ID NO: 50 - protein, 107 aa, Artificial Sequence
  antiBCMA Ab 4 VL
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 51 - protein, 122 aa, Artificial Sequence
  antiBCMA Ab 4 VH
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 52 - protein, 111 aa, Artificial Sequence
  antiBCMA Ab 5 VL
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 53 - protein, 117 aa, Artificial Sequence
  antiBCMA Ab 5 VH
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 54 - protein, 246 aa, Artificial Sequence
  anti-TACI scFv 1
DIVMTQSQKFMSTTVGDRVSITCKASQNVGTAVAWYQQKPGQSPKLLIYSASNRYTGVPDRFTGSGSGTDFTLTISNMQSEDLADYFCQQYSSYRTFGGGTKLEIKRSGGGGSGGGGSGGGGSQVTLKESGPGMLQPSQTLSLTCSFSGFSLSTFGMGVGWIRQPSGKGLEWLAHIWWDDAQYSNPALRSRLTISKDTSKNQVFLKIANVDTADTATYYCSRIHSYYSYDEGFAYWGQGTLVTVSS

SEQ ID NO: 55 - protein, 239 aa, Artificial Sequence
  anti-TACI scFv 2
DIVMTQSQKFMSTTVGDRVTITCKASQNVGTAVAWYQQKPGQSPKLLIYSASNRYTGVPVRFTGSGSGTDFTLTINNMQSEDLADYFCQQYSSYPLTFGAGTKLELKRSGGGGSGGGGSGGGGSQVQLKQSGPGLVAPSQSLSITCTVSGFSLTSYGVDWVRQSPGKGLEWLGIIWGGGRTNYNSAFKSRLSISKDNSKSQVFLKMNSLQTDDTAMYYCASGDRAADYWGQGTSVTVSS

SEQ ID NO: 56 - protein, 244 aa, Artificial Sequence
  anti-TACI scFv 3
QAVVTQESALTTSPGETVTLTCRSSTGAVTTSDYAHWVQEKPDHLFTGLIGGTNNRAPGVPARFSGSLIGDKAALTITGAQTEDEAIYFCALWYSNHWVFGGGTKLTVLSGGGGSGGGGSGGGGSEVQLVESGGGLVKPGGSLKLSCAASGFTFSDYAMSWVRQTPEKRLEWVATISDGGTYTYYPDNIKGRFTISRDNTKNNLYLQMSHLKSEDTAMYYCARYYGVYYAMGCWGQGTSVTVSS

SEQ ID NO: 57 - protein, 20 aa, Artificial Sequence
  cleavage site, 2A-like sequence
RAEGRGSLLTCGDVEENPGP

SEQ ID NO: 58 - protein, 1400 aa, Artificial Sequence
  construct Fc scFv Strand Exchange
METDTLLLWVLLLWVPGSTGDVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIKRSGGGGSGGGGSGGGGSQVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWLQGSQELPREKYLTWAPVLDSDGSFFLYSILRVAAEDWKKGDTFSCSVMHEALHNHYTQKSLDRSPGKKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAEGRGSLLTCGDVEENPGPMETDTLLLWVLLLWVPGSTGDIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIKRSGGGGSGGGGSGGGGSQVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSSDPAEPKSPDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGKKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 1394 aa, Artificial Sequence
  construct Fc scFv Knob into holes
METDTLLLWVLLLWVPGSTGDVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIKRSGGGGSGGGGSGGGGSQVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAEGRGSLLTCGDVEENPGPMETDTLLLWVLLLWVPGSTGDIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIKRSGGGGSGGGGSGGGGSQVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 1394 aa, Artificial Sequence
  construct Fc scFv Electrostatic steering
METDTLLLWVLLLWVPGSTGDVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIKRSGGGGSGGGGSGGGGSQVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYDTTPPVLDSDGSFFLYSDLTVDKSRWQQGNVFSCSVMHEALHNHYTQKSLSLSPGKKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAEGRGSLLTCGDVEENPGPMETDTLLLWVLLLWVPGSTGDIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIKRSGGGGSGGGGSGGGGSQVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSSDPAEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLTCLVKGFYPSDIAVEWESNGQPENNYKTTPPVLKSDGSFFLYSKLTVDKSRWQQGNVFSCSVMHEALHNHYTQKSLSLSPGKKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 954 aa, Artificial Sequence
  construct dual variable Fab
METDTLILWVLLLLVPGSTGDIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIKRTVAAPSVFIFPPDVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRAATNFSLLKQAGDVEENPGPMGWSCIILFLVATATGVHSQVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSSASTKGPSVFPLAPQVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPKDPKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 18 aa, Artificial Sequence
  linker
DPAEPKSPDKTHTCPPCP

SEQ ID NO: 63 - protein, 13 aa, Artificial Sequence
  linker (VH linker long)
ASTKGPSVFPLAP

SEQ ID NO: 64 - protein, 12 aa, Artificial Sequence
  linker (VL linker long)
TVAAPSVFIFPP

SEQ ID NO: 65 - protein, 6 aa, Artificial Sequence
  linker (VH linker short)
ASTKGP

SEQ ID NO: 66 - protein, 5 aa, Artificial Sequence
  linker (VL linker short)
TVAAP

SEQ ID NO: 67 - protein, 124 aa, Artificial Sequence
  antibody A7 (VHH), VH sequence
QVQLQESGGGLVQAGGSLRLSCAASGLTFSNYAMAWFRRAPGKERELVSRISGRGTLTYYADSVKGRFTISRDNDKNTVHLQMNSLKADDTAVYYCAAGSNSWGTRVVHTYDYWGQGTQVTVSS

SEQ ID NO: 68 - protein, 127 aa, Artificial Sequence
  antibody B4 (VHH), VH sequence
QVQLQQSGGGLVQAGGSLRLSCGASGRTFSSLPMAWFRQAPGKEREFVAAISGSGGATYYVDSVKGRFTISRDNAKNTVYLQMNSPKPEDTAVYYCAAKEGRFRWTYYTERFEYDSWGQGTQVTVSS

SEQ ID NO: 69 - protein, 5 aa, Artificial Sequence
  glycine-serine linker sequence
GGGGS

SEQ ID NO: 70 - protein, 5 aa, Artificial Sequence
  helical linker sequence
EAAAK

SEQ ID NO: 71 - protein, 5 aa, Artificial Sequence
  truncated COMP coiled-coil domain, C-terminal amino acids of SEQ ID NO16
CDACG

SEQ ID NO: 72 - protein, 20 aa, Artificial Sequence
  truncated COMP coiled-coil domain comprising 20 amino acids
QQVREITFLKNTVMECDACG